(19) United States
(12) Patent Application Publication
(10) Pub. No.: US 2021/0290143 A1
(43) Pub. Date: Sep. 23, 2021

(54) BIOELECTRIC CURRENT ESTIMATION METHOD, BIOELECTRIC CURRENT ESTIMATION APPARATUS, BIOMAGNETIC MEASUREMENT APPARATUS, AND BIOMAGNETIC MEASUREMENT SYSTEM

(71) Applicants: Taishi WATANABE, Tokyo (JP); Shigenori KAWABATA, Tokyo (JP); Takanori YOKOTA, Tokyo (JP); Miho AKAZA, Tokyo (JP); Hiroshi DEGUCHI, Kanagawa (JP); Sukchan KIM, Tokyo (JP); Yuki MIYANO, Tokyo (JP)

(72) Inventors: Taishi WATANABE, Tokyo (JP); Shigenori KAWABATA, Tokyo (JP); Takanori YOKOTA, Tokyo (JP); Miho AKAZA, Tokyo (JP); Hiroshi DEGUCHI, Kanagawa (JP); Sukchan KIM, Tokyo (JP); Yuki MIYANO, Tokyo (JP)

(73) Assignees: Ricoh Company, Ltd., Tokyo (JP); National University Corporation Tokyo Medical and Dental University, Tokyo (JP)

(21) Appl. No.: 17/205,136

(22) Filed: Mar. 18, 2021

(30) Foreign Application Priority Data
Mar. 23, 2020 (JP) 2020-051880
Mar. 23, 2020 (JP) 2020-051881
Nov. 13, 2020 (JP) 2020-189868

(51) Int. Cl. A61B 5/388; A61B 5/271; A61B 5/294; A61B 5/242; A61B 5/06

(52) U.S. Cl. CPC A61B 5/388; A61B 5/271; A61B 5/294; A61B 2560/04; A61B 5/066; A61B 2562/0223; A61B 2562/16; A61B 5/242

(57) ABSTRACT

A bioelectric current estimation method includes acquiring position information of a nerve in a measurement target region of a subject for which magnetic data is measured with a magnetic sensor, the position information of the nerve being acquired based on a nerve image included in a morphological image of the measurement target region; acquiring a positional relationship between a position of the nerve and a position of the magnetic sensor, based on the acquired position information of the nerve and position information of the magnetic sensor when the magnetic sensor is positioned to face the measurement target region; and estimating a neural activity current, which is generated in association with neural activity of the subject, based on the acquired positional relationship and the magnetic data of the measurement target region measured by the magnetic sensor.

### CROSS-REFERENCE TO RELATED APPLICATION

The present application is based on and claims priority under 35 U.S.C. § 119 to Japanese Patent Application No. 2020-051880, filed on Mar. 23, 2020, Japanese Patent Application No. 2020-051881, filed on Mar. 23, 2020, and Japanese Patent Application No. 2020-189868, filed on Nov. 13, 2020, the contents of which are incorporated herein by reference in their entirety.

### BACKGROUND OF THE INVENTION

### 1. Field of the Invention

The present invention relates to a bioelectric current estimation method, a bioelectric current estimation apparatus, a biomagnetic measurement apparatus, and a biomagnetic measurement system.

### 2. Description of the Related Art

As a method of examining the functions of the spinal cord, peripheral nerves, and muscles, there is known a method of measuring the magnetic field generated from a living body based on activities of these portions. For example, in a biomagnetic measurement apparatus that measures the magnetic field generated from the neck or the back of a subject under examination, the leading ends of the respective sensors in a sensor array are disposed along the curved shape of the measurement portion. Then, an X-ray image is captured from the side of the subject to acquire the positional relationship between the sensor array and the nerve (see Patent Document 1).

In another biomagnetic measurement apparatus, an ultrasound probe of an ultrasonic diagnostic apparatus is disposed in a shield room in which the biomagnetic measurement apparatus is disposed. Then, a sensor array, which is for measuring the magnetic field generated from the heart of the subject by using an ultrasonic tomography image of the heart, is disposed at an appropriate measurement position of the subject to measure the biomagnetic field (see Patent Document 2).

In yet another biomagnetic measurement apparatus, by making it possible to change the relative position between the detection target portion of a subject supported on a supporting part and a biomagnetic measuring unit, it is possible to dispose a radiation detecting unit between the supporting part and the biomagnetic measuring unit. Accordingly, it is possible to acquire the position information of the detection target portion by the radiation detecting unit while the subject is in the same posture as the posture at the time when the biomagnetic field is measured (see Patent Document 3).

In another example, by placing the lower leg in a synthetic plastic boot-shaped water bath containing hot water, and applying an ultrasound probe to the outer wall of the water bath, it is possible to acquire the cross-sectional image of the lower leg while preventing a change in the shape of the lower leg that would be caused by the pressing force of the ultrasound probe. For example, the ultrasound probe is applied to the outer wall of the water tank corresponding to a position directly marked on the body surface of the subject (see Non-patent Document 1).

Patent Document 1: Japanese Patent No. 4834076

Patent Document 2: Japanese Patent No. 3094988

Patent Document 3: Japanese Unexamined Patent Application Publication No. 2019-98156

Non-patent Document 1: Seiichi Hisamoto, Masatoshi Higuchi, “Study of an ultrasonic echo method for observing extremity cross sections using a water bath”, Journal of the Society of Biomechanisms, vol. 34, no. 1(2010)

### SUMMARY OF THE INVENTION

According to one aspect of the present invention, there is provided a bioelectric current estimation method including acquiring position information of a nerve in a measurement target region of a subject for which magnetic data is measured with a magnetic sensor, the position information of the nerve being acquired based on a nerve image included in a morphological image of the measurement target region; acquiring a positional relationship between a position of the nerve and a position of the magnetic sensor, based on the acquired position information of the nerve and position information of the magnetic sensor when the magnetic sensor is positioned to face the measurement target region; and estimating a neural activity current, which is generated in association with neural activity of the subject, based on the acquired positional relationship and the magnetic data of the measurement target region measured by the magnetic sensor.

### BRIEF DESCRIPTION OF THE DRAWINGS

FIG. 1 is a block diagram illustrating an example of a biomagnetic measurement system according to a first embodiment of the present invention;

FIG. 2 is a flow chart illustrating an example of an operation of a biomagnetic measurement system illustrated in FIG. 1 according to the first embodiment of the present invention;

FIG. 3 is a diagram illustrating an example of an ultrasound image acquired by an ultrasonic measuring unit of FIG. 1 according to the first embodiment of the present invention;

FIG. 4 is a diagram illustrating an example in which a positional relationship between a nerve and a sensor unit is acquired by a positional relationship acquiring unit of FIG. 1 according to the first embodiment of the present invention;

FIG. 5 is a diagram illustrating an example of a current waveform (timewise variation of current intensity) estimated by a current estimating unit based on magnetic field data measured by a magnetic field measuring unit according to the first and second embodiments of the present invention;

FIG. 6 is a diagram illustrating the peak intensities of the current waveforms illustrated in FIG. 5 in the order of the waveform numbers according to the first and second embodiments of the present invention;

FIG. 7 is a block diagram illustrating an example of a hardware configuration of the bioelectric current estimation apparatus of FIG. 1 according to the first embodiment of the present invention;

FIG. 8 is a block diagram illustrating an example of a biomagnetic measurement system according to a second embodiment of the present invention;

FIG. 9 is a block diagram illustrating an example in which the position of a camera in the biomagnetic measurement system of FIG. 8 is changed according to the second embodiment of the present invention;

FIG. 10 is a perspective view illustrating an example of a sliding structure of a movable plate provided in a protruding part of a Dewar of FIG. 8 according to the second embodiment of the present invention;

FIG. 11 is a perspective view illustrating another example of a movable plate provided in a protruding part of a Dewar of FIG. 8 according to the second embodiment of the present invention;

FIG. 12 is a block diagram illustrating an example of functions of the biomagnetic measurement system of FIG. 8 according to the second embodiment of the present invention;

FIG. 13 is a flow chart illustrating an example of an operation of the biomagnetic measurement system of FIGS. 8 and 9 according to the second embodiment of the present invention;

FIG. 14A is a diagram illustrating an example of an ultrasound image acquired by the ultrasonic measurement apparatus of FIGS. 8 and 9 according to the second embodiment of the present invention;

FIG. 14B is a diagram illustrating a comparison example of an ultrasound image acquired by an ultrasonic measurement apparatus according to the related art;

FIG. 15 is a diagram illustrating an example in which the positional relationship between the nerve and the magnetic sensor of the sensor array is acquired by the positional relationship acquiring unit of FIG. 12 according to the second embodiment of the present invention;

FIG. 16 is a block diagram illustrating an example of a biomagnetic measurement system according to a third embodiment of the present invention;

FIG. 17 is a perspective view illustrating an example of a movable plate disposed on a protruding part of a Dewar according to a fourth embodiment of the present invention;

FIG. 18 is a diagram illustrating an example of an evaluation result when an ultrasound image of a measurement target portion is acquired through a sensor facing region formed of various materials according to the fourth embodiment of the present invention;

FIG. 19 is a diagram illustrating an example of an ultrasound image of a measurement target portion acquired through a sensor facing region formed of a material indicated in FIG. 18 according to the fourth embodiment of the present invention;

FIG. 20 is a diagram illustrating an example of an ultrasound image of a measurement target portion acquired through a sensor facing region formed of a material indicated in FIG. 18 according to the fourth embodiment of the present invention;

FIG. 21 is a diagram illustrating an example of an ultrasound image of a measurement target portion acquired through a sensor facing region formed of a material indicated in FIG. 18 according to the fourth embodiment of the present invention;

FIG. 22 is a diagram illustrating an example of an ultrasound image of a measurement target portion acquired through a sensor facing region formed of a material indicated in FIG. 18 according to the fourth embodiment of the present invention;

FIG. 23 is a diagram illustrating an example of an ultrasound image of a measurement target portion acquired through a sensor facing region formed of a material indicated in FIG. 18 according to the fourth embodiment of the present invention;

FIG. 24 is a diagram illustrating an example of an ultrasound image of a measurement target portion acquired through a sensor facing region formed of a material indicated in FIG. 18 according to the fourth embodiment of the present invention; and

FIG. 25 is a block diagram illustrating an example of a hardware configuration of the data processing apparatus of FIGS. 8, 9, and 16 according to the second and third embodiments of the present invention.

### DETAILED DESCRIPTION OF THE PREFERRED EMBODIMENTS

In a biomagnetic measurement system such as a magnetospinography system of the conventional technology, for example, the nerve function is evaluated by estimating the electrical current distribution in the body by using an estimation technique such as spatial filtering from magnetic field data obtained by the sensor array. The magnetic field data signal changes rapidly depending on the distance between the magnetic field source and the sensor, and, therefore, it is necessary to acquire the position information of the nerve beforehand and apply the acquired position information to the estimation technique such as the spatial filtering method.

For example, when attempting to estimate the neural activity current of the spinal cord, the spinal cord is located in the spinal canal within the spine, so it is possible to acquire the positional relationship between the spinal cord and the sensor array, from the spinal canal appearing in the X-ray image. However, when attempting to estimate the activity of a nerve, such as a peripheral nerve, for which the positional relationship between the bone and the nerve is not uniquely determined, it is difficult to acquire the positional relationship between the nerve and the sensor array from an X-ray image, and it is difficult to accurately estimate the neural activity current.

A problem to be addressed by an embodiment of the present invention is to acquire the positional relationship between a sensor for measuring a biomagnetic field and a nerve by using a nerve image included in an image of the measurement target region, and to estimate the neural activity current generated in association with the neural activity of a subject under examination.

Further, an ultrasound measurement apparatus of the conventional technology can acquire ultrasound images including images of nerves. However, there is no established technique for accurately detecting the position of a subject on which an ultrasound probe is being applied. Thus, in the conventional technology, for example, the position at which the ultrasound probe is to be applied on the body surface of the subject, has been directly marked. Furthermore, when pressing the ultrasound probe against the body surface of the subject, the position of the nerve in the ultrasound image may be displaced relative to the position of the nerve at the time of acquiring the biomagnetic field.

Another problem to be addressed by an embodiment of the present invention is to estimate the current distribution from the biomagnetic field data by using an ultrasound image to enable the identification of the positional relationship between the position of the nerve and the position of the magnetic sensor at the time of magnetic field measurement.

Hereinafter, embodiments will be described with reference to the drawings. In the drawings, the same elements are denoted by the same reference numerals and overlapping descriptions may be omitted.

### First Embodiment

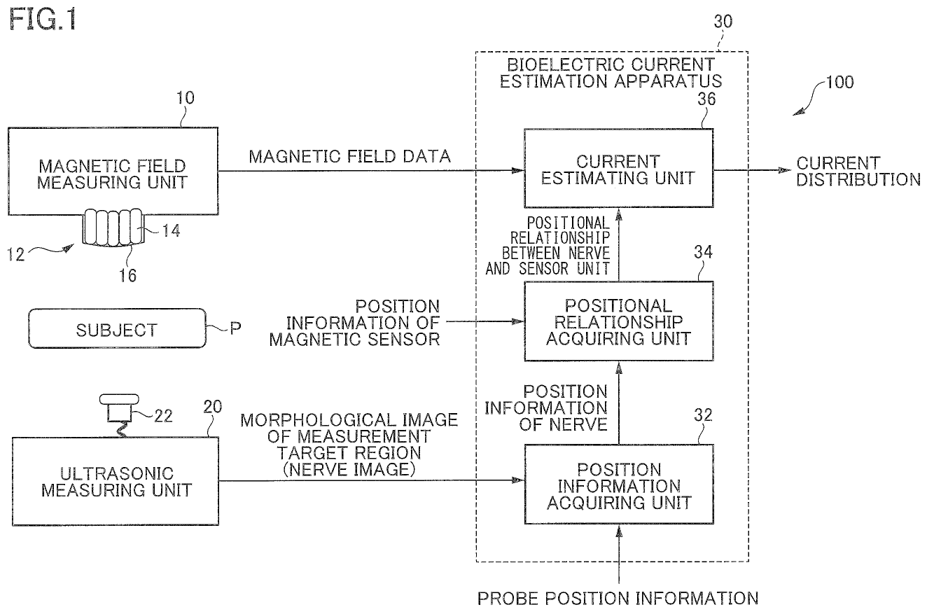

FIG. 1 is a block diagram illustrating an example of a biomagnetic measurement system according to a first embodiment of the present invention. A biomagnetic measurement system 100 illustrated in FIG. 1 includes a magnetic field measuring unit 10, an ultrasonic measuring unit 20, and a bioelectric current estimation apparatus 30. The bioelectric current estimation apparatus 30 includes a position information acquiring unit 32, a positional relationship acquiring unit 34, and a current estimating unit 36.

For example, the magnetic field measuring unit 10 is included in a biomagnetic measurement apparatus installed in a magnetic shield room (not illustrated), and the bioelectric current estimation apparatus 30 is included in a computer such as a Personal Computer (PC) or a server installed outside the magnetic shield room. The bioelectric current estimation apparatus 30 may be implemented by a bioelectric current estimation program executed by a central processing unit (CPU) installed in a computer.

The magnetic field measuring unit 10 includes a magnetic sensor 12 and a cover member 16 covering the leading end of the magnetic sensor 12.

The magnetic sensor 12 includes a plurality of sensor units 14 arranged in an array to measure the magnetic field of a subject P under examination. The cover member 16 is disposed at a position to cover the leading ends of the sensor units 14 and has a curved cross-sectional shape. Here, the plane of the diagram corresponds to the cross-sectional plane, and the depth direction of the diagram corresponds to a vertical direction. A plurality of the sensor units 14 are disposed so that the leading end positions facing the subject P are disposed along the curved shape of the cover member 16.

For example, the sensor unit 14 includes a superconducting quantum interference device (SQUID). That is, the magnetic sensor 12 is a SQUID sensor array. Hereinafter, a plurality of the sensor units 14 are also referred to as the sensor array 14. The magnetic field measuring unit 10 measures a magnetic field induced in a nerve of the measurement target of the subject P measured by electrical stimulation by a nerve stimulating apparatus (not illustrated). The magnetic field measuring unit 10 outputs the measured magnetic field to the bioelectric current estimation apparatus 30 as magnetic field data.

For example, the ultrasonic measuring unit 20 is an ultrasonic examination apparatus including an ultrasound probe 22 and may be located either inside or outside the magnetic shield room. An ultrasound image examination apparatus uses an image examination technique in which ultrasound is applied to a subject and the echoes from the subject are visualized, and, therefore, a nerve image indicating a nerve that cannot be obtained by X-ray photography can be directly visually confirmed. The ultrasonic measuring unit 20 outputs a morphological image of the subcutaneous tissue obtained by measuring the measurement target region (region to be measured), to the bioelectric current estimation apparatus 30. For example, when the ultrasound image of the subcutaneous tissue of the forearm is measured by the ultrasonic measuring unit 20, the morphological information output to the bioelectric current estimation apparatus 30 includes a nerve image of the forearm.

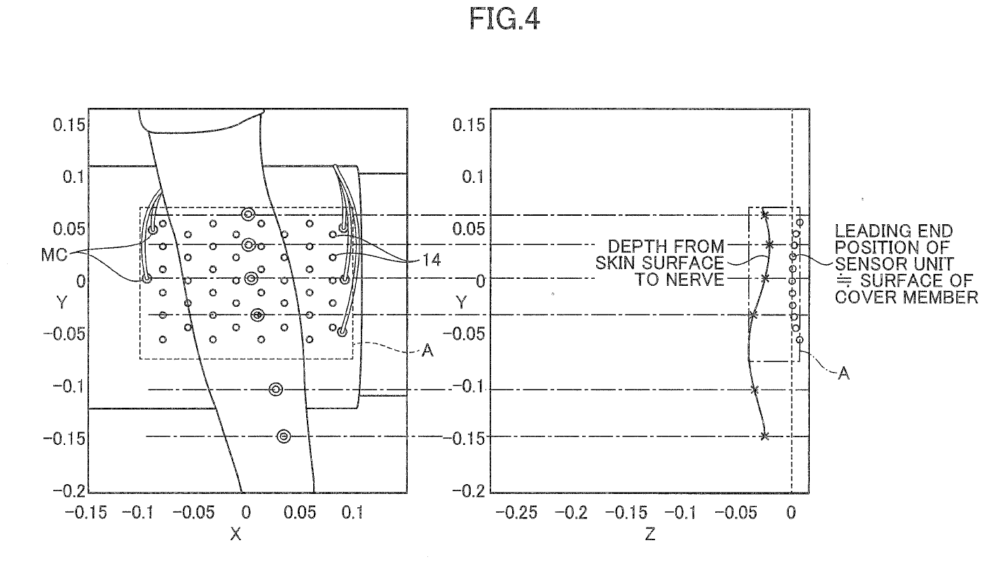

Here, the measurement target region is the region of the subject P that faces the sensor array 14, and is a region A for acquiring the magnetic field data represented by the XY coordinates on the left side of FIG. 4. Hereinafter, any position (two-dimensional position) on the measurement target region may be referred to as an XY position. In the measurement target region, a position in the direction perpendicular to the body surface of the subject P (for example, the position of the nerve in the depth direction relative to the surface of the skin) corresponds to the Z coordinate on the right side of FIG. 4, and may hereinafter be referred to as the Z coordinate position. For example, the body surface of the subject P in the measurement target region is the surface of the measurement target region.

The ultrasonic measuring unit 20 outputs ultrasonic waves from the ultrasound probe 22 toward the measurement target region of the subject P and receives echoes from the subject P, thereby acquiring an image (morphological information) of the subcutaneous tissue (muscle, nerve, bone, etc.) of the measurement target region of the subject P.

When the ultrasonic measuring unit 20 is installed in the magnetic shield room, and the magnetic field data of the subject P in the magnetic shield room is measured by the magnetic field measuring unit 10, an ultrasound image of the measurement target region of the subject P is captured by the ultrasonic measuring unit 20. On the other hand, when the ultrasonic measuring unit 20 is installed outside the magnetic shield room, for example, the ultrasound image of the measurement target region of the subject P is captured by the ultrasonic measuring unit 20 at a timing different from the timing of measuring the magnetic field data of the subject P by the magnetic field measuring unit 10.

In the present embodiment, the nerve for which the magnetic field is to be measured, is a peripheral nerve. For example, when the neural activity current generated in association with the neural activity is acquired by measuring the magnetic field data of the peripheral nerve of the forearm of the subject P, the magnetic field data is measured by the magnetic field measuring unit 10 while the cubital fossa portion of the forearm is in contact with the cover member 16. The ultrasound probe 22 is applied to (positioned to face) the cubital fossa of the forearm to acquire an ultrasound image of the subcutaneous tissue of the cubital fossa.

The biomagnetic measurement system 100 may include a magnetic resonance tomography apparatus instead of the ultrasonic measuring unit 20. In this case, the position information acquiring unit 32 receives a Magnetic Resonance (MR) image from the magnetic resonance tomography apparatus as a morphological image of a measurement target region including a nerve image, instead of an ultrasound image, and acquires the position information of the nerve.

In the bioelectric current estimation apparatus 30, the position information acquiring unit 32 receives the morphological image of the measurement target region including the nerve image from the ultrasonic measuring unit 20 and the probe position information representing the XY position of the ultrasound probe 22 when each morphological image is acquired. The probe position information includes time information. The position information acquiring unit 32 acquires the position information of the nerve (for example, the XY position and the Z position at a plurality of points on the travel path of the nerve) based on the morphological image and the probe position information. The position information acquiring unit 32 may receive a morphological image in which the probe position information is added to the morphological image acquired by the ultrasonic measuring unit 20, instead of receiving the probe position information.

The positional relationship acquiring unit 34 acquires the positional relationship between the position of the nerve and the position of each of the sensor units 14 based on the position information of the nerve acquired by the position information acquiring unit 32 and the position information of each of the sensor units 14 of the magnetic sensor 12. For example, the positional relationship acquiring unit 34 acquires the positional relationship between a plurality of points on the nerve and each of the sensor units 14 based on the position (XY position and Z position) of the plurality of points on the nerve on three-dimensional coordinates and the position (XY position and Z position) of each of the sensor units 14 on three-dimensional coordinates.

The position information of each of the sensor units 14 of the magnetic sensor 12 is acquired in advance by using design data, etc., of the magnetic field measuring unit 10. The Z position of the nerve and the Z position of each of the sensor units 14 indicate the Z coordinate value assuming that the Z position of a reference point, which is the most protruding point on the surface of the cover member 16, is set to “0”, and indicate the distance with a sign from the reference point.

The current estimating unit 36 performs arithmetic processing (computational processing) to identify the estimated current of the neural activity at a specified measurement point in the measurement target region by using an estimation algorithm such as a spatial filtering method based on the positional relationship between a plurality of points of the nerve and each of the sensor units 14 acquired by the positional relationship acquiring unit 34. The current estimating unit 36 outputs current information representing the estimated neural activity current.

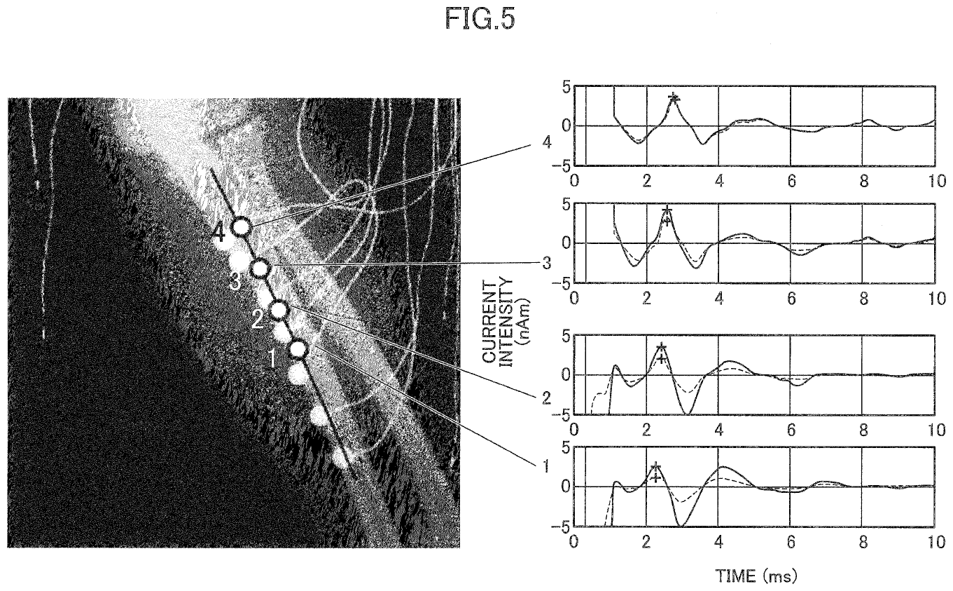

The specified measurement point in the measurement target region may be a point on the nerve or a plurality of points included in a predetermined range within the measurement target region. The estimated neural activity current is displayed on a display screen of a data processing apparatus including the bioelectric current estimation apparatus 30, as a current waveform indicative of the variation over time (timewise variation), for example, as illustrated in FIG. 5, which will be described below. When a plurality of points included within a predetermined range of the measurement target region are specified, the orientation or intensity distribution of the current flowing in the nerve and around the nerve and the timewise variation of these parameters can be displayed on a display screen.

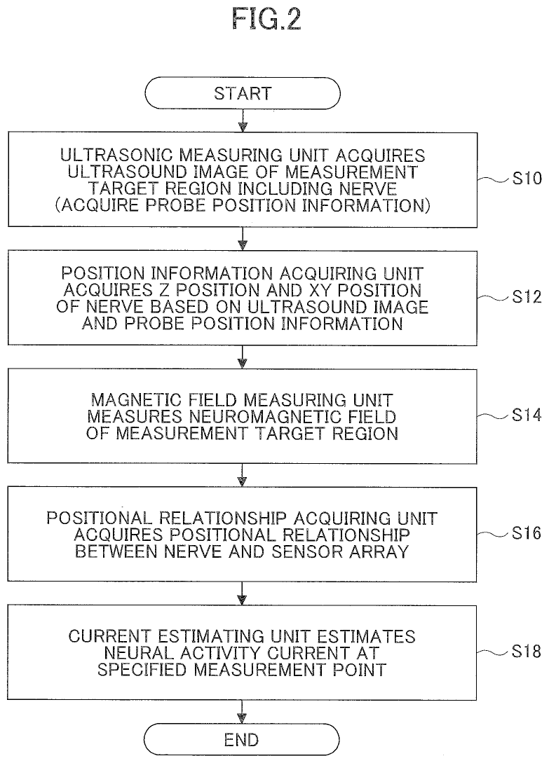

FIG. 2 is a flow chart illustrating an example of an operation of the biomagnetic measurement system 100 of FIG. 1. Steps S12, S16, and S18 of FIG. 2 indicate an example of a method for estimating a bioelectric current for estimating a neural activity current generated in association with a neural activity of a subject P based on the magnetic field data of the subject P obtained by measurement by the magnetic sensor 12 and the nerve image of the subject P. Steps S12, S16, and S18 of FIG. 2 indicate an example of a bioelectric current estimation program for estimating a neural activity current generated in association with a neural activity of the subject P based on the magnetic field data of the subject P obtained by the measurement by the magnetic sensor 12 and the nerve image of the subject P.

First, in step S10, when the ultrasound probe 22 is applied to (positioned to face) the skin of the measurement target region of the subject P and is moved along the travel direction of the nerve, the ultrasonic measuring unit 20 generates an ultrasound image (morphological image) of the measurement target region including the nerve.

At this time, probe position information indicating the trajectory where the ultrasound probe 22 has moved is recorded together with the ultrasound image. The probe position information may be extracted, for example, from an image captured by a camera from above the ultrasound probe 22 in a region that includes the measurement target region, and may be synchronized with the ultrasound image based on the time information.

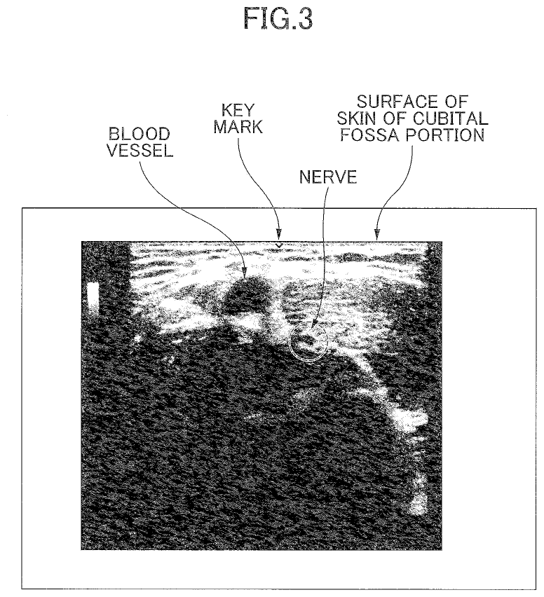

Next, in step S12, the position information acquiring unit 32 acquires a Z position and an XY position at each of a plurality of points of the nerve based on the ultrasound image. The method of acquiring the Z position of the nerve will be described with reference to FIG. 3. The position information acquiring unit 32 acquires the XY position of the nerve based on the ultrasound image and the probe position information which are associated with each other by the time information. The Z position and the XY position are the position information at a plurality of points specified at the time of acquiring the ultrasound image, or the position information at a plurality of points set at equal intervals on the travel path of the nerve set by the position information acquiring unit 32.

Next, in step S14, the magnetic field measuring unit 10 measures the neuromagnetic field of the measurement target region of the subject P. Next, in step S16, the positional relationship acquiring unit 34 acquires continuous position information (distance information), for example, by n-order function approximation, based on the position information of the nerve and the sensor array 14 acquired discretely in the measurement target region. For example, the positional relationship acquiring unit 34 acquires the positional relationship between a plurality of positions of the nerve and the positions of each of the sensor units 14 based on the position Z and the XY position of the nerve acquired by the position information acquiring unit 32 and the position information of each of the sensor units 14 of the magnetic sensor 12 acquired in advance.

Next, in step S18, the current estimating unit 36 estimates the neural activity current at a specified measurement point using, for example, a spatial filtering method based on the positional relationship between a plurality of positions of the nerve and the positions of each of the sensor units 14 acquired by the positional relationship acquiring unit 34. The estimated neural activity current is displayed, for example, as a current waveform or current intensity map, on a display screen of a data processing apparatus including the bioelectric current estimation apparatus 30.

FIG. 3 is a diagram illustrating an example of an ultrasound image acquired by the ultrasonic measuring unit 20 of FIG. 1. As described above, the ultrasound image is acquired by moving the ultrasound probe 22 along the travel direction of the nerve while the ultrasound probe 22 is applied to (positioned to face) the surface of the skin of the measurement target region of the subject P.

The ultrasound image illustrated in FIG. 3 has been acquired at a certain position on the forearm of the subject P, which is the measurement target region, and the upper side of FIG. 3 illustrates the surface of the skin of the cubital fossa. In the present embodiment, for example, approximately five ultrasound images are acquired to calculate the timewise variation of the estimated activity current of the nerve and the conduction velocity of the electrical signal within the nerve, and the like, and FIG. 3 is one of these ultrasound images. In the ultrasound image, subcutaneous tissues such as the nerves, blood vessels, and muscles, etc., are appearing, and, therefore, the positional relationship of these tissues and the distance of these tissues and from the surface of the skin can be acquired.

In each ultrasound image, the distance (depth) from the skin surface to the nerve can be measured by using a distance measurement function by specifying two positions in the ultrasound image.

With respect to the position of the nerve in the ultrasound image, the positional relationship with the blood vessel and the positional relationship with the skin are clear for each measurement target region (e.g., forearm). Furthermore, the cross-sectional shape of the nerve in the ultrasound image is unique for each measurement target region. Therefore, the distance (depth) from the skin surface to the nerve may be obtained by image analysis of the ultrasound image to determine the position of the skin surface and the position of the nerve. In this case, a mechanical learning method, such as deep learning, may be used.

It is also clear where the ultrasound probe 22 is applied to in the measurement target region while the ultrasound probe 22 is acquiring the ultrasound image. As described with reference to FIG. 2, probe position information (XY position of the ultrasound probe 22) can be acquired by photographing by a camera. Further, in the upper center of the ultrasound image, a key mark is displayed to indicate the center position of the ultrasound probe 22. Therefore, the XY position of the nerve in the measurement target region can be determined based on the probe position information and the ultrasound image. The probe position information may be acquired by acquiring the ultrasound image by the ultrasonic measuring unit 20 while marking the position of the ultrasound probe 22 on the skin and then capturing the measurement target region including the mark with a camera.

Note that FIG. 3 illustrates an example in which an ultrasound image including a nerve image is acquired by applying an ultrasound probe 22 to the skin surface of the anterior side (palm side) of the elbow portion of the subject P, but an ultrasound image including a nerve image may be acquired by applying the ultrasound probe 22 to the skin surface of the posterior side (dorsal side) of the elbow portion.

FIG. 4 is a diagram illustrating an example in which the positional relationship between the nerve and the sensor unit 14 is acquired by the positional relationship acquiring unit 34 in FIG. 1. The image on the left side of FIG. 4 is a morphological image including the measurement target region A in which the cubital fossa of the forearm of the subject P is placed on the cover member 16 of the magnetic field measuring unit 10. In the image, the elbow portion of the forearm is visible, the upper side of the image is the upper arm side, and the lower side of the image is the wrist side. The image on the left in FIG. 4 is an image in which the measurement target region A, the XY position of the nerve, and the XY position of the sensor array 14 are superimposed on an image taken by the camera.

The small circles dispersed in a staggered manner indicate the XY positions of the sensor units 14 of the magnetic field measuring unit 10. A plurality of double circles indicate the XY positions of the nerve and are acquired by the position information acquiring unit 32. The positions of the double circles are also the positions where the ultrasound images have been acquired by the ultrasound probe 22.

In the measurement target region A, a plurality of markers MC are disposed on the outside of the left and right of the region where the sensor units 14 are disposed. The marker MC is a coil photographed together with the subject P to associate the morphological image, such as an X-ray image, with the measurement position of the magnetic field data measured by the magnetic sensor 12, and a predetermined current is applied to the coil.

The positional relationship between the marker MC and each of the sensor units 14 is acquired in advance. Therefore, by identifying the positions of the markers MC by the magnetic sensor 12, it is possible to detect that each of the sensor units 14 is positioned at a small circle in the drawing. The dashed-dotted line extending laterally from the left image to the right graph in FIG. 4 indicates the position where the ultrasound image is acquired by the ultrasonic measuring unit 20 in FIG. 1.

The graph on the right side of FIG. 4 illustrates the Z position (depth) of the nerve from the body surface of the anterior side (palm side) of the elbow portion of the subject P in the measurement target region A and the surrounding area thereof, corresponding to the image on the left side. A small circle on the right indicates the Z position (depth) of the leading end of the sensor unit 14. The curve obtained by connecting the small circles on the right side indicates the position of the surface of the cover member 16.

When the biomagnetic field is measured by the magnetic field measuring unit 10, the anterior side (the palm side) of the elbow portion of the subject P is brought in contact with the cover member 16. Thus, in the right graph, the distance between the Z position of a curve obtained by connecting the circles and the Z position of the nerve indicates the distance from the surface of the skin to the nerve. A value obtained by adding the distance from the surface of the skin to the nerve and the distance from the surface of the cover member 16 to the leading end of the sensor unit 14, represents the distance from the leading end of the sensor unit 14 to the nerve.

The surface of the cover member 16 and the plane connecting the leading ends of the sensor units 14 have a curved cross-section, and, therefore, the Z position of the nerve is corrected according to the difference between the Z position of the nerve and the Z position of the leading end of the sensor unit 14, rather than using the “0” position of the Z coordinate as a reference. In this case, correction of the Z position of the nerve may be performed in consideration of the gap between the surface of the cover member 16 and the leading end of the sensor unit 14 and the thickness of the cover member 16.

The current estimating unit 36 estimates the neural activity current at a specified measurement point (in the example of FIG. 4, a double circle) based on the XY position of the nerve and the

XY position of each of the sensor units 14 illustrated on the left side of FIG. 4, and the Z position of the nerve and the Z position of each of the sensor units 14 illustrated on the right side of FIG. 4.

FIG. 5 is a diagram illustrating an example of a current waveform (timewise variation of current intensity) estimated by the current estimating unit 36 based on magnetic field data measured by a magnetic field measuring unit 10 in FIG. 1. The left side of FIG. 5 illustrates an image in which the travel direction of the nerve is superimposed on an X-ray image of the forearm of the subject P as a matter of facilitating the explanation, and is not used for estimating the neural activity current by the current estimating unit 36. The lower side of the X-ray image is the wrist.

The current waveform of the solid line on the right side of FIG. 5 illustrates the timewise variation of the current intensity estimated in consideration of the Z position (distance from the skin (depth)) of the nerve acquired by the position information acquiring unit 32. The current waveform of the dashed line on the right side of FIG. 5 illustrates the timewise variation (comparison example) of the current intensity estimated assuming that the Z position of the nerve is constant.

The timewise variation of the current intensity illustrated in FIG. 5 is estimated from the biomagnetic field generated by the current flowing through the nerve axon in response to electrical stimulation when a nerve stimulating apparatus is applied to the wrist side of the forearm. The current is transmitted from the distal side (lower side of FIG. 5) to the proximal side (upper side of FIG. 5) of the nerve axon. Thus, in both the current waveform of the solid line and the current waveform of the dashed line, the time when the peak intensity of the current appears becomes delayed as the position of the nerve is closer to the proximal side. The waveform number of the current waveform is represented by 1 to 4 from the distal side to the proximal side.

Further, in terms of neurophysiology, the peak intensity of the current waveform is almost constant or decreases as the position is closer to the proximal side that is far from the position where the electrical stimulation is applied. However, the peak intensity of the current waveform of the dashed line becomes smaller as the position is closer to the distal side, and, therefore, the current intensity is not estimated correctly. This is because, when estimating the current intensity assuming that the Z position of the nerve is constant, an error occurs with respect to the depth of the actual nerve, and this error appears as an error when executing an arithmetic process to estimate the current intensity from the magnetic field data.

On the other hand, the peak intensity of the current waveform of the solid line estimated based on the Z position of the actual nerve does not appreciably change, and it can be determined that the current intensity is correctly estimated by an arithmetic process using an estimation algorithm such as the spatial filtering method.

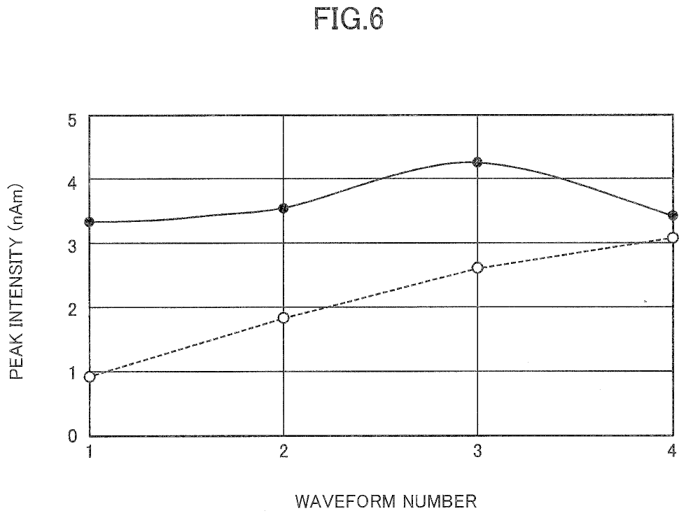

FIG. 6 is an explanatory diagram illustrating the peak intensities of the current waveforms illustrated in FIG. 5 in the order of the waveform numbers. The solid line indicates the characteristic of the current intensity estimated in consideration of the Z position of the nerve acquired by the position information acquiring unit 32. The dashed line indicates the characteristic (comparison example) of the current intensity estimated by assuming that the Z position of the nerve is constant. In the solid-line characteristic, the rate of variation of the current intensity according to the waveform number indicating the position of the nerve is small. On the other hand, in the dashed-line characteristic, the rate of variation of the current intensity according to the waveform number is large. That is, in the biomagnetic measurement system 100 illustrated in FIG. 1, the current intensity can be estimated more accurately by considering the distance from the skin surface to the nerve that is the Z position.

Note that also when a plurality of points for estimating the current intensity are provided in the measurement target region A, and the direction of the current or the intensity distribution of the current flowing in the nerve and around the nerve is estimated, the current intensity can be correctly estimated, similar to the case described with reference to FIGS. 5 and 6.

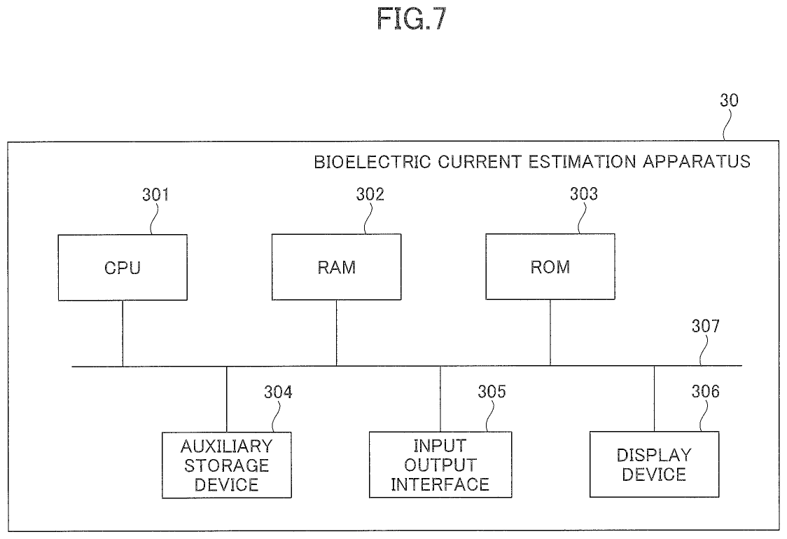

FIG. 7 is a block diagram illustrating an example of a hardware configuration of the bioelectric current estimation apparatus 30 of FIG. 1. For example, the bioelectric current estimation apparatus 30 is a data processing apparatus (information processing apparatus) and includes a central processing unit (CPU) 301, a random access memory (RAM) 302, a read-only memory (ROM) 303, an auxiliary storage device 304, an input output interface 305, and a display device 306, which are interconnected by a bus 307.

The CPU 301 controls the overall operation of the bioelectric current estimation apparatus 30. The CPU 301 implements the function of the position information acquiring unit 32, the positional relationship acquiring unit 34, and the current estimating unit 36 by executing a bioelectric current estimation program stored in the ROM 303 or the auxiliary storage device 304. The CPU 301 may control the operation of the biomagnetic measurement system 100, such as the magnetic field measuring unit 10 and the ultrasonic measuring unit 20.

The RAM 302 is used as a work area of the CPU 301 and stores a bioelectric current estimation program and various parameters such as the Z position and the XY position. The ROM 303 stores a bioelectric current estimation program.

The auxiliary storage device 304 is a storage device such as a Solid State Drive (SSD) or a Hard Disk Drive (HDD). For example, the auxiliary storage device 304 stores a control program, such as an operating system (OS) for controlling the operation of the bioelectric current estimation apparatus 30, an ultrasound image, morphological image data, various parameters, and the like.

The input output interface 305 is coupled to a mouse, keyboard, or the like. The input output interface 305 may include a communication interface for communicating with other devices. The display device 306 displays windows for displaying the current waveforms illustrated in FIG. 5 and operation windows. The display device 306 may display an ultrasound image illustrated in FIG. 3 or a figure illustrating the positional relationship between the nerve and the sensor unit 14 illustrated in FIG. 4.

Thus, in the present embodiment, the position of the nerve relative to the sensor array 14 can be accurately acquired by using an image (an ultrasound image or a Magnetic Resonance (MR) image) that includes a nerve image in which the nerve is directly captured, as compared to indirectly acquiring the position of the nerve from an X-ray image. For example, by using the actual XY position and Z position of the nerve acquired by using an ultrasound image or an MR image, the positional relationship with the XY position and the Z position of each of the sensor units 14 can be accurately acquired. Because the position of the nerve can be accurately obtained, for example, by an arithmetic process using a spatial filtering method, the intensity of the current flowing through the nerve can be correctly estimated, and the accuracy of estimating the current intensity can be improved.

When acquiring the position Z of the nerve, by correcting the Z position of the nerve according to the shape of the surface of the cover member 16 and the plane obtained by connecting the leading ends of the sensor units 14, the positional relationship between the XY position and the Z position of the nerve and the XY position and the Z position of each of the sensor units 14 can be accurately acquired. Thus, the intensity of the current flowing through the nerve can be estimated even more correctly, and the accuracy in estimating the current intensity can be further improved.

### Second Embodiment

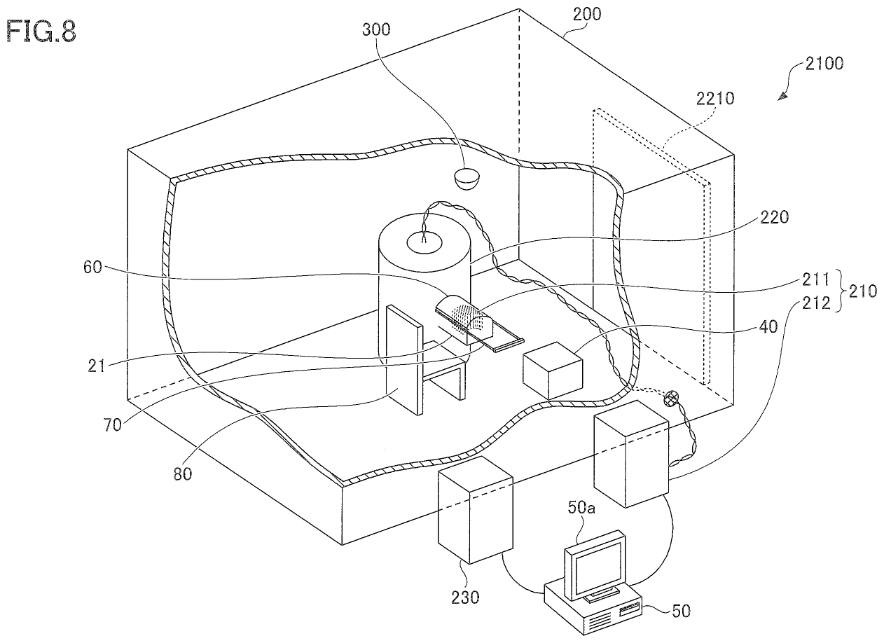

FIG. 8 is a system configuration diagram illustrating an example of a biomagnetic measurement system according to a second embodiment of the present invention. A biomagnetic measurement system 2100 illustrated in FIG. 8 includes a magnetic measurement apparatus 210, a Dewar 220, a nerve stimulation apparatus 230, an ultrasonic measurement apparatus 40, and a data processing apparatus 50. The magnetic measurement apparatus 210 is an example of a biomagnetic measurement apparatus.

The magnetic measurement apparatus 210 includes a sensor array 211 including a plurality of superconducting quantum interference devices (SQUID) and a signal processing apparatus 212. The magnetic measurement apparatus 210 is coupled to the data processing apparatus 50, and the operation thereof is controlled by the data processing apparatus 50. The data processing apparatus 50 includes a computer, such as a server or a Personal Computer (PC), which can execute various kinds of data processes by executing programs.

The sensor array 211 is housed within a protruding part 21 protruding from the Dewar 220. For example, the top surface of the protruding part 21 has a curved cross-sectional shape. The magnetic detecting units at the leading ends of the respective magnetic sensors (SQUID sensors) of the sensor array 211, are positioned facing and along the curved-shape inner surface of the protruding part 21. The protruding part 21 is an example of a sensor housing unit in which magnetic sensors of the sensor array 211 are housed.

A guide rail 70 disposed along the protruding direction of the protruding part 21 is fixed on the protruding part 21. The guide rail 70 is an example of a guide member. A movable plate 60 is slidably mounted to the guide rail 70 in the protruding direction of the protruding part 21 (the right lower direction in the figure, the orthogonal direction of the cross-section). For example, the movable plate 60 has a curved shape corresponding to the curved shape of the top surface of the protruding part 21 and is movable on the guide rail 70 to slide along the top surface of the protruding part 21. The movable plate 60 is an example of a plate member, and the upper surface of the protruding part 21 is an example of an opposing surface facing the magnetic sensor.

For example, the movable plate 60 is a resin plate, for example, made of polyethylene terephthalate, and uses a plate material that is transparent to visible light and passes a magnetic field. It is preferable that the movable plate 60 is colorless and transparent. Preferably, the material of the movable plate 60 is a non-magnetic material and has an acoustic impedance that is close to that of the human body. The movable plate 60 may be made of polycarbonate other than polyethylene terephthalate. The movable plate 60 is set to have a thickness (e.g., approximately 1 mm to 5 mm, preferably approximately 1 mm to 2 mm) so as not to deflect when the ultrasound probe is pressed against the movable plate 60. The movable plate 60 may be thicker at the peripheral portions (on the sides corresponding to the guide rail 70) that requires more strength compared to the central portion.

Mounting the guide rail 70 to the protruding part 21 facilitates the construction of a mechanism for moving the movable plate 60, as compared to a mechanism for moving the movable plate 60 together with a chair or bed. Therefore, the guide rail can be easily mounted to the magnetic measurement apparatus 210 which is already in operation, and the magnetic measurement apparatus 210 can be converted into an apparatus which can acquire ultrasound images with minimal cost.

The nerve stimulation apparatus 230 applies electrical stimulation to the subject via an electrode attached to the body surface of the subject to induce the neural activity of the subject. The nerve stimulation apparatus 230 is coupled to the data processing apparatus 50 and the operation thereof is controlled by the data processing apparatus 50. In FIG. 8, the illustrations of a cable coupled to the nerve stimulation apparatus 230 and an electrode to apply electrical stimulation to the subject are omitted. The data processing apparatus 50 may operate the nerve stimulation apparatus 230 and the magnetic measurement apparatus 210 in synchronization with each other.

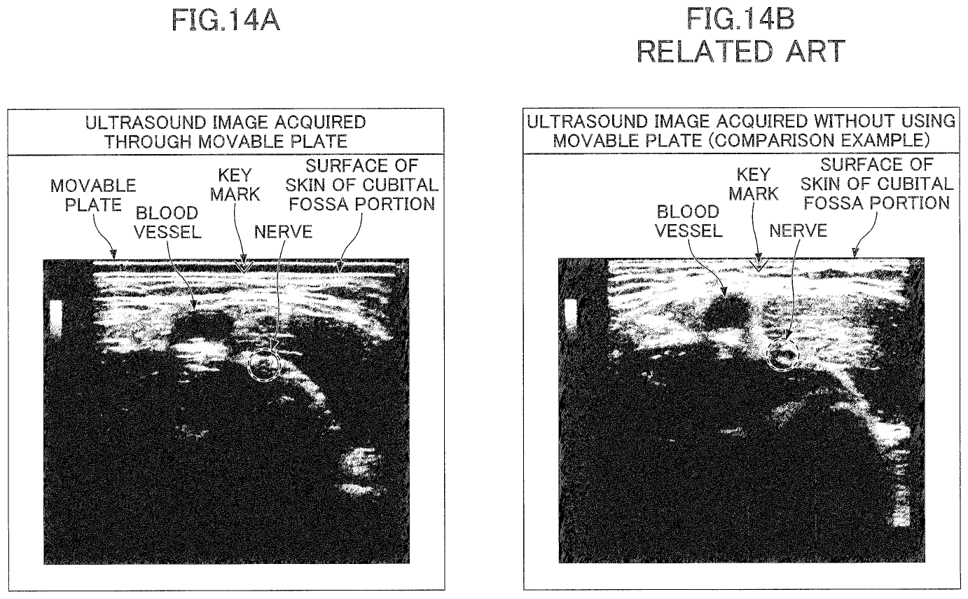

The ultrasonic measurement apparatus 40 acquires an ultrasound image of the measurement target region before the magnetic measurement apparatus 210 measures the biomagnetic field of the subject. As will be described with reference to FIGS. 14A and 14B, an ultrasound image makes it possible to identify the position of the nerve because the nerve appears in the ultrasound image. The ultrasonic measurement apparatus 40 is coupled to the data processing apparatus 50, and an ultrasound image acquired by the ultrasonic measurement apparatus 40 can be transferred to the data processing apparatus 50 and can be displayed on a display device 50a of the data processing apparatus 50.

The Dewar 220, which includes the protruding part 21, and the ultrasonic measurement apparatus 40 are disposed within a magnetic shield room 200 that shields the magnetism. The magnetic shield room 200 has an interior space, for example, which is approximately 2.5 m wide and 2.5 m high, and approximately 3 m long, and has a door 2210 for conveying objects such as the Dewar 220 and for the entry of people.

Within the magnetic shield room 200, a chair 80 may be disposed near the protruding part 21 for seating the subject. In the present embodiment, the subject sitting on the chair 80 places his or her forearm on the upper surface of the movable plate 60 so that the anterior side (palm side) of the elbow portion contacts the movable plate 60 and the elbow portion faces the upper surface of the protruding part 21 via the movable plate 60. At this time, the movable plate 60 is drawn toward the Dewar 220.

The ceiling of the magnetic shield room 200 is provided with a camera 300 above the movable plate 60. The camera 300 may be a video camera capable of capturing videos or a digital still camera capable of capturing still images. When the ultrasound image of a subject is acquired by the ultrasonic measurement apparatus 40, the camera 300 captures the measurement target portion (forearm) of the subject placed on the movable plate 60 drawn out along the guide rail 70, and an ultrasound probe (not illustrated) visible through the movable plate 60.

The camera 300 is coupled to the data processing apparatus 50. An image acquired by the camera 300 can be transferred to the data processing apparatus 50 and can be displayed on the display device 50a. Further, if the camera 300 is a digital still camera, a release operation of the camera 300 may be performed by an operator operating the ultrasound probe. Accordingly, with the digital still camera, it is possible to acquire an image at any timing intended by the operator (i.e., the timing at which an appropriate ultrasound image is acquired).

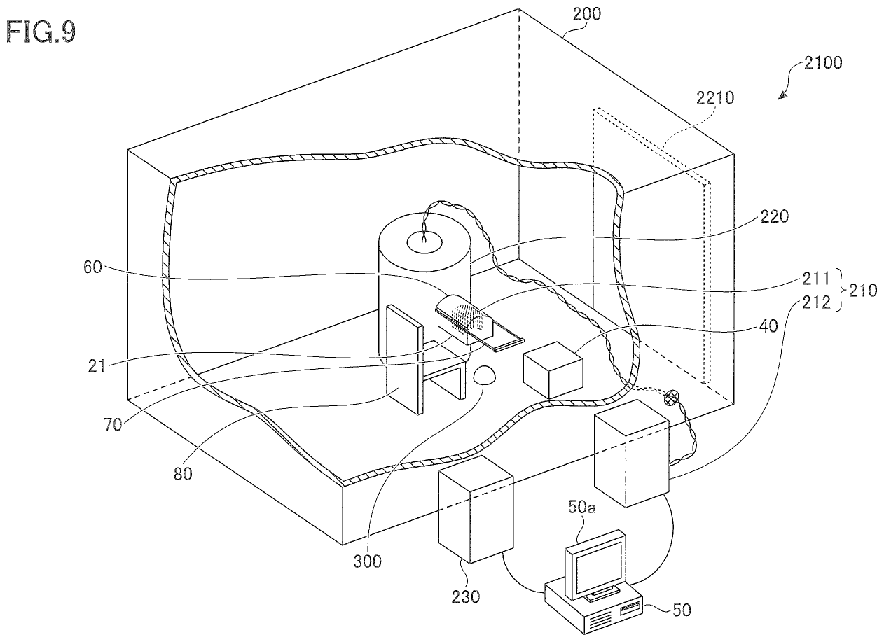

FIG. 9 is a diagram illustrating an example in which the position of the camera 300 is changed in the biomagnetic measurement system of FIG. 8. In FIG. 9, the camera 300 is located on the floor below the protruding part 21, rather than on the ceiling of the magnetic shield room 200. The camera 300 is mounted on the floor to capture the measurement portion (measurement position) of a subject to which an ultrasound probe is applied through the transparent movable plate 60.

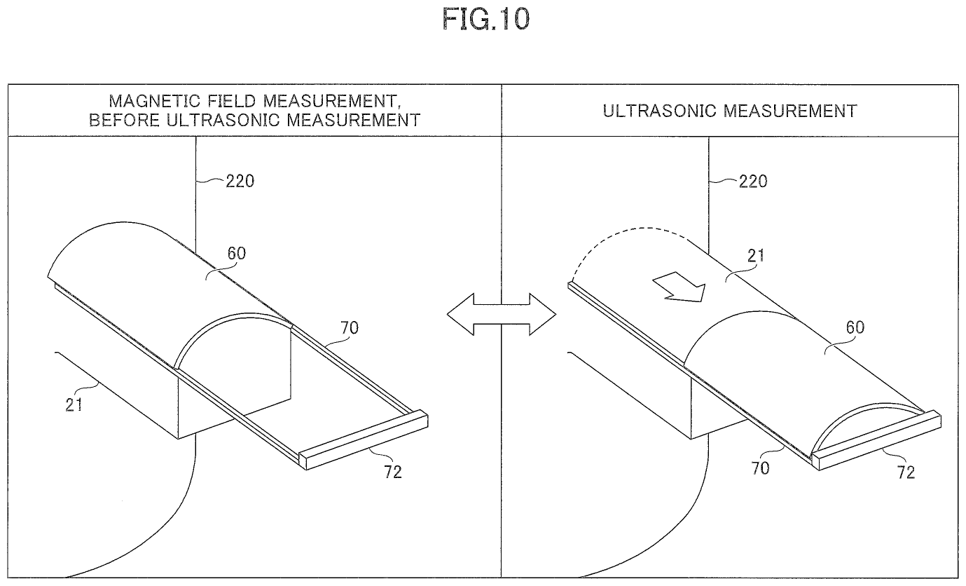

FIG. 10 is a perspective view illustrating an example of a slide structure of the movable plate 60 provided in the protruding part 21 of the Dewar 220 of FIG. 8. In FIG. 10, the subject (forearm) is omitted. The movable plate 60 is retracted toward the Dewar 220 at the time of the measurement of the biomagnetic field by the magnetic measurement apparatus 210 (FIG. 8) and before the ultrasonic measurement by the ultrasonic measurement apparatus 40 (FIG. 8).

While the movable plate 60 is retracted toward the Dewar 220, the subject sits in the chair (FIG. 8), and places his or her forearm placed on the protruding part 21 (on the movable plate 60) with the anterior side (the palm side) of the elbow portion in contact with the movable plate 60. This is the state when the biomagnetic field is measured by the magnetic measurement apparatus 210.

Then, while the anterior side (the palm side) of the elbow portion is in contact with the movable plate 60, the movable plate 60 is moved while sliding on the guide rail 70 and is pulled away to the side opposite the Dewar 220. In this manner, the movable plate 60 can be separated from the top surface of the protruding part 21. The movable plate 60 is moved against a stopper 72 attached to the leading end of the guide rail 70. For example, the movable plate 60 is fixed to the stopper 72 by a locking mechanism, such as a latch (not illustrated), with the leading end of the movable plate 60 positioned against the stopper 72.

The movable plate 60 merely moves while the arm is placed thereon, and, therefore, the contact state of the anterior side (palm side) of the elbow portion with respect to the movable plate 60 when the movable plate 60 is fixed to the stopper 72, is the same as that when the movable plate 60 is retracted toward the Dewar 220. That is, the contact state of the anterior side (the palm side) of the elbow portion with the movable plate 60 is the same as the state when the biomagnetic field is measured by the magnetic measurement apparatus 210. With the movable plate 60 fixed to the stopper 72, the ultrasound probe of the ultrasonic measurement apparatus 40 is applied to the back side (lower side) of the movable plate 60, and an ultrasound image is acquired at a plurality of points of the measurement target portion of the subject via the movable plate 60.

At this time, a pressing force caused by the leading end of the ultrasound probe is applied to the back surface of the movable plate 60, but the movable plate 60 is not flexed because the movable plate 60 is stiff, so the contact state of the anterior side (palm side) of the elbow portion to the movable plate 60 is not changed. Therefore, an ultrasound image can be acquired by maintaining the shape of the measurement target portion of the subject to be the same as that when a biomagnetic field is measured by the magnetic measurement apparatus 210.

Here, the measurement target portion of the subject being the same shape, means that position of the nerve of the measurement target portion is the same at the time of acquiring the ultrasound image and at the time of measuring the biomagnetic field. The position of the nerve in the measurement target portion includes the position in the plane direction facing the movable plate 60 and the position in the depth direction from the movable plate 60. Hereinafter, the position in the plane facing the movable plate 60 is also referred to as the XY position, and the depth from the movable plate 60 (skin) is also referred to as the Z position.

Because the movable plate 60 is transparent, the operator of the ultrasound probe can recognize the position of the ultrasound probe without peering into the back side of the movable plate 60. Because the movable plate 60 is transparent, the position of the ultrasound probe can be captured by the camera 300 mounted on the ceiling of the magnetic shield room 200 while the movable plate 60 is drawn out, and the image captured by the camera 300 can be recognized.

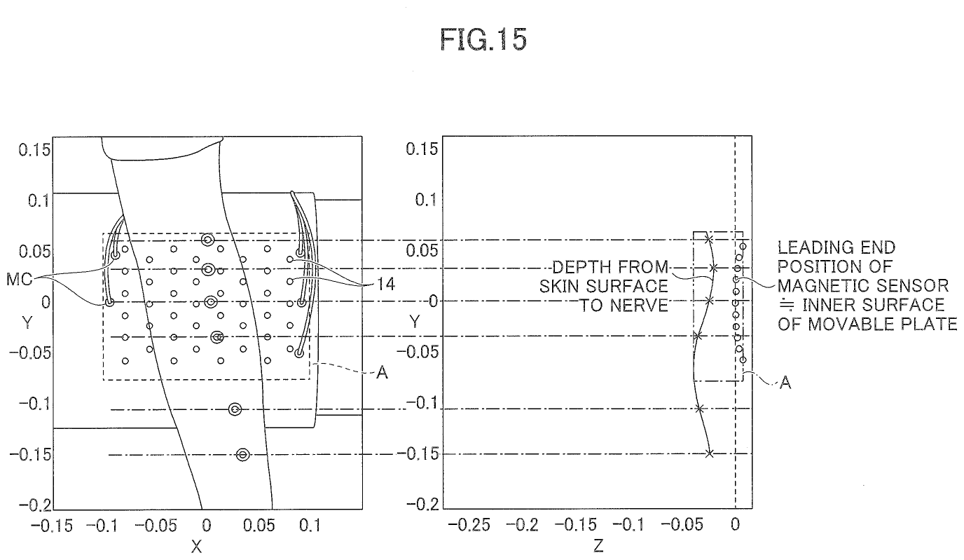

The movable plate 60 and the stopper 72 are unlocked with respect to each other, after the ultrasound image has been acquired and position information indicating the position of the nerve in the measurement target region of the subject has been acquired. The acquisition of the position information of the nerve is described in FIG. 15. The ultrasound image acquired by the ultrasonic measurement apparatus 40 and the image captured by the camera 300 are transferred to the data processing apparatus 50.

Then, while the anterior side (palm side) of the elbow portion is in contact with the movable plate 60, the movable plate 60 is retracted toward the Dewar 220 along the guide rail 70, to return to the state illustrated on the left side of FIG. 10. The movable plate 60 may then be fixed to the outer wall surface of the Dewar 220 by, for example, a locking mechanism.

In this state, electrical stimulation is applied to the subject from the nerve stimulation apparatus 230 (FIG. 8), and the magnetic field generated from the nerve in the elbow portion, which is the measurement target, is measured by the magnetic measurement apparatus 210. The magnetic measurement apparatus 210 transfers the biomagnetic field data representing the measured magnetic field to the data processing apparatus 50. Preferably, the electrode that applies electrical stimulation to the subject is pre-attached to the subject prior to acquiring the ultrasound image.

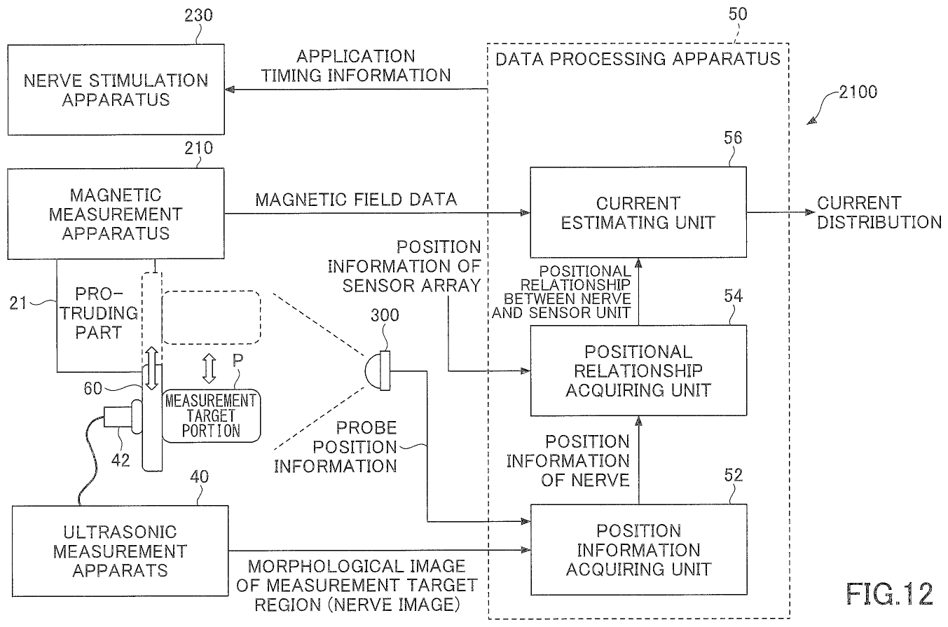

The data processing apparatus 50 estimates the neural activity current based on the position information of the nerve obtained from the ultrasound image and the image captured by the camera 300, the position information of the magnetic sensor, and the biomagnetic field data measured by the magnetic measurement apparatus 210. The data processing apparatus 50 displays a current waveform, etc., representing the estimated current, on the display device 50a. The process of estimating the neural activity current by the data processing apparatus 50 will be described with reference to FIG. 12.

In FIGS. 8 and 10, an example in which a space where the ultrasound probe enters, is provided under the movable plate 60 by moving the movable plate 60 in a horizontal direction along the horizontally extending guide rail 70 mounted to the protruding part 21, to acquire an ultrasound image, is described. However, a vertically extending guide rail may be attached to the Dewar 220 to move the movable plate 60 in a vertical direction along the guide rail. In this case, the movable plate 60 is moved upward until there is a space where the ultrasound probe can enter under the movable plate 60, and then an ultrasound image is acquired.

When the movable plate 60 is mounted to the Dewar 220 in a manner as to be movable in the vertical direction, ultrasonic imaging and measurement of the biomagnetic field may be performed while the subject is standing, without providing a chair in the magnetic shield room 200. A chair which moves vertically in conjunction with the vertical movement of the movable plate 60 may be disposed within the magnetic shield room 200. In this case, the movable plate 60 may be mounted together with the chair on a moving mechanism that moves vertically, and may be moved together with the chair. Then, ultrasonic imaging and measurement of the biomagnetic field are performed while the subject is seated in a the chair.

Further, in FIG. 8, the chair 80 may be fixed to a movable mechanism horizontally movable in conjunction with the horizontal movement of the movable plate 60, to move the chair 80 together with the movement of the movable plate 60. In this case, it is possible to further prevent a change in the shape (position of the nerve) when the anterior side (palm side) of the elbow portion is in contact with the movable plate 60. The movable plate 60, or a horizontally or vertically movable mechanism including the movable plate 60, may be moved by an electric operation rather than being manually moved.

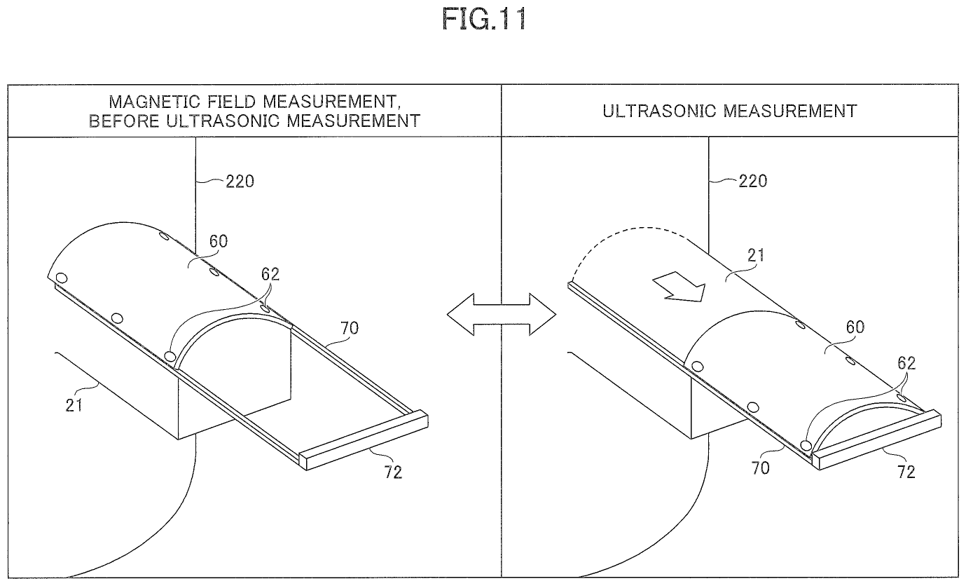

As illustrated in FIG. 11, a marker coil 62 that generates magnetism may be provided on the movable plate 60 and at least one magnetic sensor may be provided on an ultrasound probe (not illustrated). The magnetic field generated by the marker coil 62 may be detected by the magnetic sensor of the ultrasound probe, and the positional relationship between the ultrasound probe and the movable plate 60 may be calculated based on the detected magnetic field data. In this case, based on the positional relationship between the nerve that is the measurement target and the sensor array 211, obtained from the ultrasound image (the image of the nerve) obtained by the ultrasound probe and the positional relationship between the ultrasound probe and the movable plate 60, it is possible to estimate the current distribution within the measurement target portion from the biomagnetic field data of the measurement target portion measured by the sensor array 211, without installing the camera 300 illustrated in FIGS. 8 and 9.

FIG. 12 is a block diagram illustrating an example of functions of the biomagnetic measurement system 2100 of FIG. 8. As described above, the ultrasonic measurement apparatus 40 acquires an ultrasound image of the measurement target portion of the subject P by an ultrasound probe 42 which is placed in a space formed under the movable plate 60 (left in FIG. 12) while the movable plate 60 is moved to the outside of the protruding part 21. The ultrasonic measurement apparatus 40 outputs the acquired ultrasound image as a morphological image (including a nerve image) of a measurement target region in the biomagnetic field, to the data processing apparatus 50.

At the time of acquiring an ultrasound image, the camera 300 captures the movable plate 60 and the surrounding area thereof to acquire an image of a measurement target portion (e.g., the forearm) of the subject P, the movable plate 60, and the ultrasound probe visible through the movable plate 60. The camera 300 outputs the acquired image as probe position information indicating the position of the ultrasound probe to the data processing apparatus 50. The camera 300 may be installed on the floor of the magnetic shield room 200 as illustrated in FIG. 9.

The nerve stimulation apparatus 230 receives application timing information indicative of the application timing of the electrical stimulation from the data processing apparatus 50 and generates the electrical stimulation. The magnetic measurement apparatus 210 measures the magnetic field induced in the nerve in the measurement target portion of the subject P in response to the electrical stimulation from the nerve stimulation apparatus 230. The magnetic measurement apparatus 210 outputs the measured magnetic field as magnetic field data to the data processing apparatus 50.

The data processing apparatus 50 includes a position information acquiring unit 52, a positional relationship acquiring unit 54, and a current estimating unit 56. For example, the position information acquiring unit 52, the positional relationship acquiring unit 54, and the current estimating unit 56 function as a biomagnetic current estimation apparatus for estimating the neural activity current based on magnetic field data, etc., measured by the magnetic measurement apparatus 210. The position information acquiring unit 52, the positional relationship acquiring unit 54, and the current estimating unit 56 may be implemented by a biomagnetic current estimation program executed by a Central Processing Unit (CPU) installed in the data processing apparatus 50.

The position information acquiring unit 52 receives a morphological image (ultrasound image) of a measurement target region including a nerve image received from the ultrasonic measurement apparatus 40 and probe position information including an image representing the XY position of the ultrasound probe 42 captured by the camera 300 when the morphological image is acquired. The probe position information includes time information. The position information acquiring unit 52 acquires the position information of the nerve (for example, the XY position and the Z position at a plurality of points on the travel path of the nerve) based on the morphological image and the probe position information.

Note that as illustrated in FIG. 11, when the marker coil 62 is installed on the movable plate 60 and a magnetic sensor is installed in the ultrasound probe, the position information acquiring unit 52 acquires the probe position information based on the magnetic field data detected by the magnetic sensor of the ultrasound probe instead of the probe position information from the camera 300. The position information acquiring unit 52 acquires the position information of the nerve based on the ultrasound image including the nerve image received from the ultrasonic measurement apparatus 40 and the probe position information.

The positional relationship acquiring unit 54 calculates the positional relationship information representing the positional relationship between the position of the nerve and the position of each magnetic sensor based on the position information of the nerve acquired by the position information acquiring unit 52 and the position information of each magnetic sensor of the sensor array 211. For example, the positional relationship acquiring unit 54 calculates the positional relationship information between a plurality of points on the nerve and each magnetic sensor based on the position (XY position and Z position) of a plurality of points on the nerve on the three-dimensional coordinates and the position (XY position and Z position) of each magnetic sensor on the three-dimensional coordinates.

The position information of each magnetic sensor is acquired in advance using design data or the like of the magnetic measurement apparatus 210. For example, the Z position of the nerve and the Z position of each of the sensor units 14 indicate a Z coordinate value assuming that the Z position of a reference point, which is the most protruding point among the leading ends of the magnetic sensors facing the inner surface of the curved shape of the protruding part 21, is set to “0”, and indicate the distance with a sign from the reference point.

The current estimating unit 56 estimates the neural activity current at a specified measurement point in the measurement target region using an estimation algorithm such as a spatial filtering method based on the positional relationship information between a plurality of points on the nerve and each magnetic sensor acquired by the positional relationship acquiring unit 54. The current estimating unit 56 outputs current information (current distribution) representing the estimated neural activity current.

The measurement point specified in the measurement target region may be a point of the nerve or a plurality of points included in a predetermined range within the measurement target region. The estimated neural activity current is displayed on the display device 50a of the data processing apparatus 50 as a current waveform indicative of timewise variation, for example, as illustrated in FIG. 5. When a plurality of points included within a predetermined range of the measurement target region are specified, the orientation and intensity of the current flowing in the nerve and around the nerve and the timewise variation of these parameters can be displayed on the display device 50a.

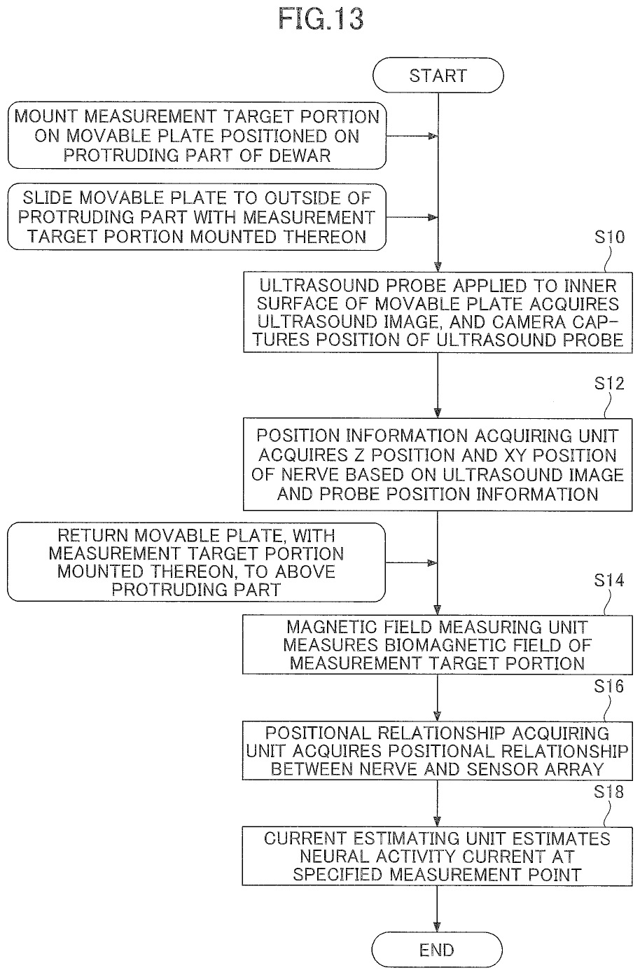

FIG. 13 is a flow chart illustrating an example of an operation of the biomagnetic measurement system 2100 of FIGS. 8 and 9. Steps S12, S16, and S18 of FIG. 13 indicate an example of a bioelectric current estimating method for estimating the neural activity current generated in association with the neural activity of the subject P based on the magnetic field data of the subject P obtained by measuring the biomagnetic field by the sensor array 211 and a nerve image of the subject P. Steps S12, S16, and S18 of FIG. 13 indicate an example of a bioelectric current estimating program for estimating the neural activity current generated in association with the neural activity of the subject P based on the magnetic field data of the subject P obtained by measuring the biomagnetic field by the sensor array 211 and a nerve image of the subject P

Before starting the operation of FIG. 13, the measurement target portion of the subject P is mounted on the movable plate 60 which is retracted toward the protruding part 21 of the Dewar 220. For example, the measurement target portion is the elbow portion. Then, while the anterior side (palm side) of the elbow portion is in contact with the movable plate 60, and the movable plate 60 is slid to the outside of the protruding part 21.

In this state, at step S10, the ultrasound probe 42 is applied to the inner surface of the movable plate 60 and an ultrasound image of the measurement target portion of the subject P is acquired through the movable plate 60. The camera 300 captures an image indicating the positional relationship between the ultrasound probe 42 and the movable plate 60 at the time when the ultrasound image is acquired.

The ultrasound image is acquired in step

S10 by controlling the ultrasonic measurement apparatus 40 by the data processing apparatus 50. The position information of the ultrasound probe 42 is acquired in step S10 by controlling the camera 300 by the data processing apparatus 50. The data processing apparatus 50 associates the position information of the ultrasound probe 42 with the ultrasound image, based on the time information output from the camera 300 together with the image and the time information included in the ultrasound image data.

If the marker coil 62 is mounted on the movable plate 60 and a magnetic sensor is mounted in the ultrasound probe 42, instead of the photographing by the camera 300, the magnetic sensor of the ultrasound probe 42 detects the magnetic field generated by the marker coil 62 mounted on the movable plate 60. The acquisition of position information of the ultrasound probe 42 in step S10 is performed based on the magnetic field detected by the magnetic sensor of the ultrasound probe 42. The data processing apparatus 50 associates the position information of the ultrasound probe 42 with respect to the movable plate 60 with the ultrasound image, based on the time information included in the ultrasound image data output from the ultrasonic measurement apparatus 40.

Next, in step S12, the position information acquiring unit 52 acquires the Z position and the XY position of a plurality of points of the nerve, respectively. The method of acquiring the Z position of the nerve will be described with reference to FIGS. 14A and 14B. The position information acquiring unit 52 acquires the XY position of the nerve based on the ultrasound image and the probe position information associated with each other by the time information.

The Z position and the XY position are the position information of a plurality of points specified at the time of acquisition of the ultrasound image.

After step S12, the movable plate 60 on which the measurement target portion is mounted is returned onto the protruding part 21. Step S12 may be performed after the movable plate 60 is returned onto the protruding part 21 or may be performed while the movable plate 60 is being returned onto the protruding part 21. Step S12 may be performed after step S14 and before performing step S16.

Next, in step S14, the magnetic measurement apparatus 210 measures the neuromagnetic field of the measurement target portion of the subject P. Next, in step S16, the positional relationship acquiring unit 54 acquires continuous position information (distance information), for example, by n-order function approximation, based on the position information of the nerve and the sensor array 211 acquired discretely in the measurement target region. For example, the positional relationship acquiring unit 54 receives the Z position and the XY position of the nerve acquired by the position information acquiring unit 52 and the position information of each magnetic sensor of the sensor array 211 previously acquired. Then, the positional relationship acquiring unit 54 acquires positional relationship information representing the positional relationship between a plurality of positions of the nerve and the position of each magnetic sensor of the sensor array 211 based on the received Z position and the XY position and the position information of each magnetic sensor.

Next, in step S18, the current estimating unit 56 estimates the neural activity current at the specified measurement point using, for example, a spatial filtering method based on the positional relationship information between a plurality of positions of the nerve and the positions of each magnetic sensor acquired by the positional relationship acquiring unit 54. The estimated neural activity current is displayed on the display device 50a of the data processing apparatus 50, for example, as a current waveform or a current intensity map.

FIGS. 14A and 14B are diagrams for describing an example of an ultrasound image acquired by the ultrasonic measurement apparatus 40 of FIGS. 8 and 9. FIG. 14A illustrates an ultrasound image acquired through the movable plate 60 with the ultrasound probe 42 applied to the inner surface (upper side in FIG. 14A) of the movable plate 60. FIG. 14B illustrates an ultrasound image (comparison example) acquired by directly applying the ultrasound probe 42 to the measurement target portion (skin) without the movable plate 60.

The ultrasound image illustrated in each of FIGS. 14A and 14B has been acquired at a position of the forearm of the subject P, which is the measurement target region, and the upper side of FIGS. 14A and 14B illustrates the surface of the skin on the anterior side (palm side) of the elbow portion. In the present embodiment, for example, approximately five ultrasound images are acquired to calculate the timewise variation of the estimated activity current of the nerve and the conduction velocity of the electrical signal within the nerve, and the like, and FIG. 14A illustrates one of such images. In ultrasound images, subcutaneous tissues such as nerves, blood vessels, and muscles appear, so that the positional relationship of these tissues and the distance from the surface of the skin can be acquired.

For example, in an ultrasound image, the distance (depth) from the surface of the skin to the nerve can be measured by a distance measuring function of the ultrasonic measurement apparatus 40 by specifying two positions in the ultrasound image. With respect to the position of the nerve in an ultrasound image, the positional relationship with a blood vessel and the positional relationship with the skin are clear for each measurement target region (e.g., forearm). Furthermore, the cross-sectional shape of the nerve in an ultrasound image is unique for each measurement target region.

However, when the ultrasound probe 42 is pressed against the skin to acquire an ultrasound image, the distance of the nerve from the skin surface varies depending on the extent of the pressing force or the like on the skin. Also, when the ultrasound probe 42 is pressed against the skin, the position of the nerve and the positional relationship the nerve and the blood vessel change. However, by applying the ultrasound probe 42 to the skin surface through the movable plate 60, the distance from the skin surface to the nerve, the position of the nerve, and the positional relationship between the nerve and the blood vessel can be made the same as when measuring the biomagnetic field. Therefore, for example, by performing image analysis of ultrasound images to obtain the position of the skin surface and the position of the nerve, the distance (depth) from the surface of the skin to the nerve can be obtained. In this case, a machine learning method, such as deep learning, may be used.

While the ultrasound image is being acquired by the ultrasound probe 42, the position where the ultrasound probe 42 is being applied in the measurement target region can be determined by analyzing an image acquired by the camera 300 with the data processing apparatus 50. That is, by the image acquired by the camera 300, probe position information (the XY position of the ultrasound probe 42) can be acquired.

Further, in the upper center of the ultrasound image, there is displayed a key mark indicating the center position of the ultrasound probe 42. Therefore, based on the probe position information and the ultrasound image, the data processing apparatus 50 can acquire the XY position of the nerve in the measurement target region.

FIG. 14A illustrate an example in which an ultrasound image including a nerve image is acquired by applying the ultrasound probe 42 through the movable plate 60 to the skin surface of the anterior side (palm side) of the elbow portion of the subject P. However, ultrasound images including nerve images may be acquired by applying the ultrasound probe 42 through the movable plate 60 to the skin surface on the posterior side (dorsal side) of the elbow portion.

FIG. 15 is a diagram illustrating an example in which the positional relationship between the nerve and each of the magnetic sensors of the sensor array 211 is acquired by the positional relationship acquiring unit 54 in FIG. 12. The image on the left side of FIG. 15 is a morphological image including the measurement target region A in which the elbow portion of the subject P is placed on the protruding part 21 of the magnetic measurement apparatus 210. In the image, the elbow portion of the forearm is visible, the upper side of the image is the upper arm side, and the lower side of the image is the wrist side. The image on the left in FIG. 15 is an image in which the measurement target region A, the XY position of the nerve, and the XY position of each of the magnetic sensors of the sensor array 211 are superimposed on an image taken by the camera 300. Note that when the marker coil 62 is installed on the movable plate 60 and the magnetic sensor is installed in the ultrasound probe 42, the image on the left side of FIG. 15 is an image in which the measurement target region A, the XY position of the nerve, and the XY position of each magnetic sensor of the sensor array 211 are superimposed.

The small circles dispersed in a staggered manner indicate the XY positions of each of the magnetic sensors of the sensor array 211. A plurality of double circles indicate the XY positions of the nerve and are acquired by the position information acquiring unit 52. The positions of the double circles are included in the positions where the ultrasound images have been acquired by the ultrasound probe 42.

In the measurement target region A, a plurality of markers MC are disposed on the outside of the left and right of the region where the sensor array 211 is disposed. The marker MC is a coil photographed together with the subject P to associate the morphological image, such as an X-ray image, with the measurement position of the magnetic field data measured by the sensor array 211, and a predetermined current is applied to the coil.

The positional relationship between the marker MC and the sensor array 211 is acquired in advance. Therefore, by identifying the positions of the markers MC by each of the magnetic sensors, it is possible to detect that each of the magnetic sensors is positioned at a small circle in the drawing. The dashed-dotted line extending laterally from the left image to the right graph in FIG. 15 indicates the position where the ultrasound image is acquired by the ultrasonic measurement apparatus 40 in FIG. 8.

The graph on the right side of FIG. 15 illustrates the Z position (depth) of the nerve from the surface of the anterior side (palm side) of the elbow portion of the subject P in the measurement target region A and the surrounding area thereof, corresponding to the image on the left side. A small circle on the right indicates the Z position (depth) of the leading end of the magnetic sensor. The curve obtained by connecting the small circles on the right side indicates the position of the surface of the protruding part 21 and the inner surface of the movable plate 60. In the right graph, the distance between the Z position of a curve obtained by connecting the circles and the Z position of the nerve indicates the distance from the magnetic sensor to the nerve.

The Z position of the nerve obtained from the ultrasound image acquired through the movable plate 60 is the same as the Z position of the nerve when measuring the biomagnetic field. Therefore, by acquiring the ultrasound image through the movable plate 60, the relationship between the Z position of the nerve illustrated on the right side of FIG. 15 and the Z position of each magnetic sensor can be obtained. That is, it is possible to obtain the relationship between the Z position of the actual nerve when measuring the biomagnetic field and the Z position of each magnetic sensor.

The movable plate 60 and the plane connecting the leading ends of the magnetic sensors have a curved cross-section, and, therefore, the Z position of the nerve is calculated by using the Z position of the leading end of the magnetic sensor as the reference, rather than using the “0” position of the Z coordinate as a reference. The current estimating unit 56 estimates the neural activity current at a specified measurement point (in the example of FIG. 15, double circles illustrated on the left side) based on the XY position of the nerve and the XY position of each magnetic sensor illustrated on the left side of FIG. 15, and the Z position of the nerve and the Z position of each magnetic sensor illustrated on the right side of FIG. 15.

FIG. 5 is a diagram illustrating an example of a current waveform (timewise variation of current intensity) estimated by the current estimating unit 56 based on magnetic field data measured by the magnetic measurement apparatus 210 in FIGS. 8 and 9. The left side of FIG. 5 illustrates an image in which the travel direction of the nerve is superimposed on an X-ray image of the forearm of the subject P as a matter of facilitating the explanation, and is not used for estimating the neural activity current by the current estimating unit 56. The lower side of the X-ray image is the wrist.

The current waveform of the solid line on the right side of FIG. 5 illustrates the timewise variation of the current intensity estimated in consideration of the Z position (distance from the skin (depth)) of the nerve acquired by the position information acquiring unit 52. The current waveform of the dashed line on the right side of FIG. 5 illustrates the timewise variation (comparison example) of the current intensity estimated assuming that the Z position of the nerve is constant.

The timewise variation of the current intensity illustrated in FIG. 5 is estimated from the biomagnetic field generated by the current flowing through the nerve axon in response to electrical stimulation when a nerve stimulating apparatus is applied to the wrist side of the forearm. The current is transmitted from the distal side (lower side of FIG. 5) to the proximal side (upper side of FIG. 5) of the nerve axon. Thus, in both the current waveform of the solid line and the current waveform of the dashed line, the time when the peak intensity of the current appears becomes delayed as the position of the nerve is closer to the proximal side. The waveform number of the current waveform is represented by 1 to 4 from the distal side to the proximal side.

Further, in terms of neurophysiology, the peak intensity of the current waveform is almost constant or decreases as the position is closer to the proximal side that is far from the position where the electrical stimulation is applied. However, the peak intensity of the current waveform of the dashed line becomes smaller as the position is closer to the distal side, and, therefore, the current intensity is not estimated correctly. This is because, when estimating the current intensity assuming that the Z position of the nerve is constant, an error occurs with respect to the depth of the actual nerve, and this error appears as an error when estimating the current intensity from the magnetic field data.

On the other hand, the peak intensity of the current waveform of the solid line estimated based on the Z position of the actual nerve does not appreciably change, and it can be determined that the current intensity is correctly estimated. As described with reference to FIG. 15, by acquiring an ultrasound image through the movable plate 60, a relationship between the Z position of the actual nerve when measuring the biomagnetic field and the Z position of each magnetic sensor can be obtained.

Therefore, the current estimating unit 56 can estimate the correct current intensity based on the correct Z position of the nerve and obtain the current waveform represented by the solid line.

FIG. 6 is an explanatory diagram illustrating the peak intensities of the current waveforms illustrated in FIG. 5 in the order of the waveform numbers. The solid line indicates the characteristic of the current intensity estimated in consideration of the Z position of the nerve acquired by the position information acquiring unit 52. The dashed line indicates the characteristic (comparison example) of the current intensity estimated by assuming that the Z position of the nerve is constant. In the solid-line characteristic, the rate of variation of the current intensity according to the waveform number indicating the position of the nerve is small. On the other hand, in the dashed-line characteristic, the rate of variation of the current intensity according to the waveform number is large. That is, in the biomagnetic measurement system 2100 illustrated in FIG. 8, by using the movable plate 60, the same position (Z position) of the nerve as when measuring the biomagnetic field can be obtained from the ultrasound image, and the accuracy of estimating the current intensity can be improved as represented by the solid line.

Note that also when a plurality of points for estimating the current intensity are provided in the measurement target region A, and the direction of the current or the intensity distribution of the current flowing in the nerve and around the nerve is estimated, the current intensity can be correctly estimated, similar to the case described with reference to FIGS. 5 and 6.

Thus, in the present embodiment, the use of a transparent movable plate 60 allows, for example, an operator of the ultrasound probe 42 to recognize the position of the ultrasound probe 42 without peering into the back side of the movable plate 60. Further, the camera 300 located on the ceiling or floor of the magnetic shield room 200 allows the ultrasound probe 42 to be photographed through the movable plate 60. Then, from the image obtained by photographing, the XY position of the ultrasound probe 42 with respect to the movable plate 60 when the ultrasound image is acquired can be detected.

Accordingly, it is possible to detect the Z position and XY position of the nerve when measuring the biomagnetic field, based on the ultrasound image and the image acquired by the camera 300. The relationship between the position of the movable plate 60 when measuring a biomagnetic field and the position of each magnetic sensor of the sensor array 211 has been acquired in advance. Therefore, the positional relationship between the position of the nerve (the Z position and the XY position) and the position of each magnetic sensor (the Z position and the XY position) can be detected, and the current distribution of the measurement target portion can be estimated from the biomagnetic field data based on the positional relationship. That is, an ultrasound image including a nerve image can be used to estimate the electrical current distribution in an living body from biomagnetic field data of peripheral nerves.

The movable plate 60 is separably disposed on the protruding part 21, so that an ultrasound image of the measurement target portion can be acquired by maintaining the measurement target portion in the same state as that when measuring the biomagnetic field. Accordingly, the position information acquiring unit 52 can detect the same position of the nerve as that when measuring the biomagnetic field even when the target measurement portion is moved to a position different from that when measuring the biomagnetic field. Therefore, the positional relationship acquiring unit 54 can accurately detect the positional relationship between the position of the nerve and the position of each magnetic sensor, and estimate the current distribution in a living body with less error from the biomagnetic field data of the peripheral nerve.

Mounting the guide rail 70 to the protruding part 21 facilitates the construction of a mechanism for moving the movable plate 60, as compared to a mechanism for moving the movable plate 60 together with a chair or bed. Therefore, the guide rail can be easily mounted to the magnetic measurement apparatus 210 which is already in operation, and the magnetic measurement apparatus 210 can be converted into an apparatus which can acquire ultrasound images with minimal cost.

### Third Embodiment

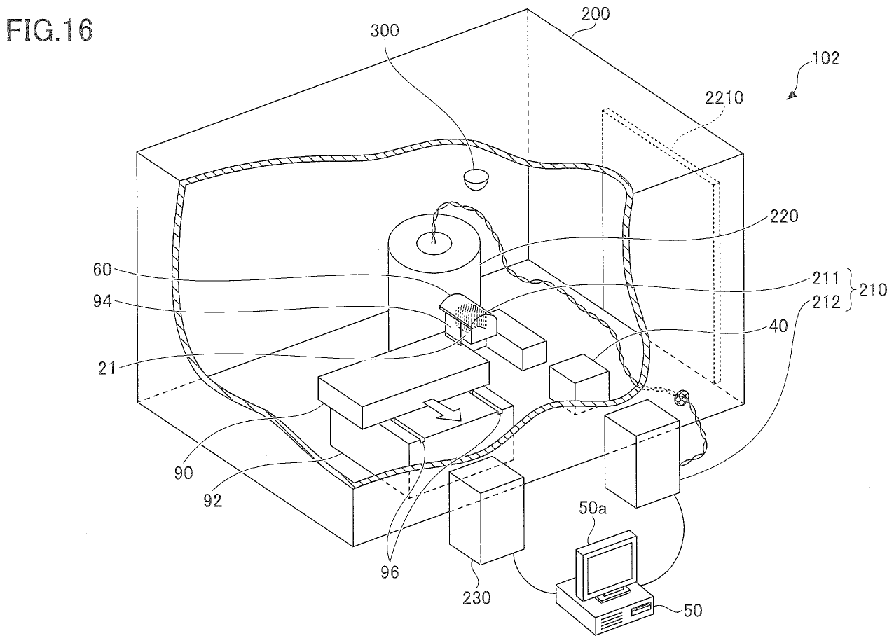

FIG. 16 is a diagram illustrating an example of a biomagnetic measurement system according to a third embodiment of the present invention. For elements similar to those in FIGS. 8 and 9, the same reference numerals shall be used, and detailed descriptions thereof shall be omitted. A biomagnetic measurement system 102 illustrated in FIG. 16 has a configuration similar to that of the biomagnetic measurement system 2100 of FIG. 8, except that a bed 90 and a pedestal 92 are included instead of the chair 80 of FIG. 8, and guide grooves 96 are included instead of the guide rail 70. In the biomagnetic measurement system 102, similarly to FIG. 9, the camera 300 may be installed below the protruding part 21 on the floor of the magnetic shield room 200.

On the bed 90, a subject (not illustrated) lies down, for example, in a supine position with his or her head at the lower left side of FIG. 16. The subject lies down in a supine position on the bed 90 with his or her knee portion placed on the movable plate 60. That is, in the present embodiment, the biomagnetic field generated from the nerve in the knee portion is measured, and the neural activity current in the knee portion is estimated based on the measured magnetic field data. The shape, structure, and mutual positional relationship of the protruding part 21 and the movable plate 60 are similar to those of FIGS. 8 and 10.

However, in the present embodiment, in the movable plate 60, both sides in the width direction of the curved cross-sectional shape (the portions mounted to the guide rail 70 of FIG. 8) are fixed to the bed 90 via a plate-like fixing member 94. The pedestal 92 includes the guide grooves 96 into which protrusions provided on the underside of the bed 90 are fitted. The guide grooves 96 are formed along a direction in which the protruding part 21 protrudes, and the bed 90 is movable in the protruding direction of the protruding part 21. The guide grooves 96 are an example of a guide member. The movable plate 60 moves together with the movement of the bed 90, so the movable plate 60 is movable in the same manner as illustrated in FIG. 8.

An ultrasound image of the knee portion is acquired by the ultrasonic measurement apparatus 40 with the bed 90 moved to the opposite side of the

Dewar 220 and the movable plate 60 moved to the outside of the protruding part 21. At this time, similar to the above-described embodiment, an image indicating the positional relationship between the ultrasound probe and the movable plate 60 is captured by the camera 300. Thereafter, the bed 90 is moved toward the Dewar 220 until the movable plate 60 is positioned on the protruding part 21 (on the sensor array 211), and the biomagnetic field in the knee portion is measured by the magnetic measurement apparatus 210.

The function of the biomagnetic measurement system 102 is similar to that of FIG. 12, except that the movable plate 60 of FIG. 12 moves in conjunction with the bed 90. Further, the operation of the biomagnetic measurement system 102 and the acquired images, waveform, and the like are the same as those of FIGS. 5, 6, and 13 to 15.

As described above, in the present embodiment, the same effect as in the above-described embodiment can be achieved. Further, in the present embodiment, a mechanism for moving the movable plate 60 with the bed 90 is provided, and therefore, it is possible to further accurately acquire the relationship between the position of the nerve in the knee portion of the subject and the position of the sensor array 211 using ultrasound images, as compared to the case where the movable plate 60 is not used. This allows a more accurate estimation of the neural activity current of the knee portion based on the measurement of the biomagnetic field generated by the nerve of the knee portion, compared to the case where the movable plate 60 is not used.

In FIG. 16, an example in which the movable plate 60 can be moved horizontally with the bed 90 has been described. However, a mechanism for vertically moving the bed 90 may be provided on the pedestal 92 to allow the movable plate 60 to be moved vertically with the bed 90. In this case, the movable plate 60 is moved upward until there is a space for the ultrasound probe to enter below the movable plate 60, and then an ultrasound image of the lower knee portion is acquired.

### Fourth Embodiment

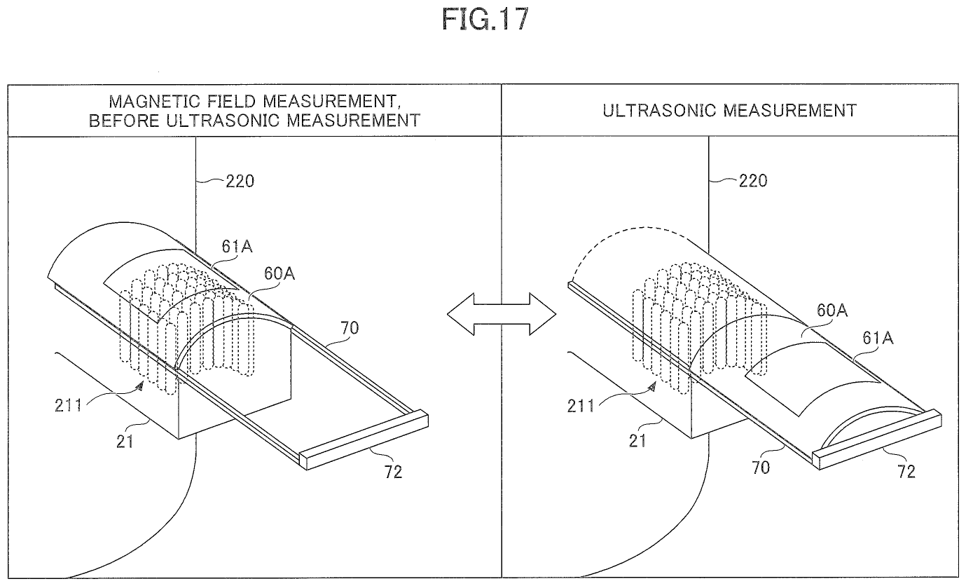

FIG. 17 is a perspective view illustrating an example of a movable plate disposed in a protruding part of a Dewar in a biomagnetic measurement system according to a fourth embodiment of the present invention. For elements similar to the above-described embodiments, the same reference numerals shall be used, and detailed descriptions thereof shall be omitted.

A movable plate 60A illustrated in FIG. 17 is slidably mounted to the guide rail 70 instead of the movable plate 60 illustrated in FIGS. 8, 9 and 16. The biomagnetic measurement system according to the present embodiment is similar to the biomagnetic measurement systems 2100 and 102 illustrated in FIGS. 8, 9 and 16, except for the structure of the movable plate 60A. Similar to FIG. 11, the marker coil 62 may be provided on the movable plate 60A.

The movable plate 60A includes a sensor facing region 61A positioned at the center of the movable plate 60A, which has a different configuration from that of the sensor facing region of the movable plate 60 illustrated in FIG. 10 or the like. The sensor facing region 61A is provided at a position facing the leading end of the sensor array 211 in a state where the movable plate 60A retracted toward the Dewar 220.

The sensor facing region 61A is the region in which the ultrasound probe 42 of the ultrasonic measurement apparatus 40 (FIG. 12) is applied from the backside (lower side) when the movable plate 60A is pulled out along guide rail 70. Note that as long as the sensor facing region 61A is provided in the region to which the ultrasound probe 42 is applied, the sensor facing region 61A may be smaller than the region of the portion facing the sensor array 211.

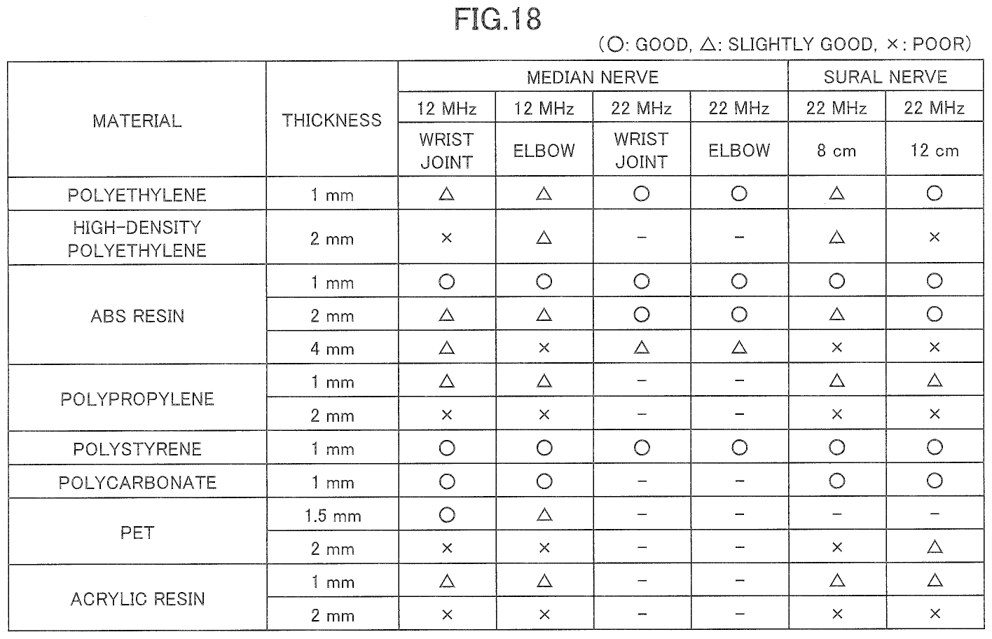

The sensor facing region 61A of the movable plate 60A is formed of a material or is formed to have a shape that facilitates the acquisition of an ultrasound image with the ultrasound probe 42. For example, the thickness of the sensor facing region 61A of the movable plate 60A is preferably less than the thickness of the region of the movable plate 60A surrounding the sensor facing region 61A. Examples of the materials and thicknesses of the sensor facing region 61A are indicated in FIG. 18.

Note that the material and the thickness of the region of the movable plate 60A surrounding the sensor facing region 61A may different from that of the sensor facing region 61A, provided that the material of the surrounding region has a rigidity that does not deform with respect to the weight of the measurement target portion of the subject P mounted on the movable plate 60A. In this case, the material of the region surrounding the sensor facing region 61A in the movable plate 60A is preferably transparent to visible light.

FIG. 18 is a diagram illustrating an example of an evaluation result when an ultrasound image of a measurement target region is acquired through the sensor facing region 61A of FIG. 17 formed of various materials. FIG. 18 illustrates an example in which ultrasound images of the median nerve at the wrist and elbow and ultrasound images of the sural nerve at a position spaced apart from the ankle at a length of 8 cm and 12 cm, respectively, have been acquired. Note that the median nerve is thicker than the sural nerve.

12 MHz and 22 MHz indicate the frequency of the ultrasound probe 42. In the ultrasound probe 42, as the frequency decreases, the resolution will be reduced, but it will be easier to reach deep portions in the body. Ultrasound images are acquired by placing the measurement target portion that is the upper limb or the lower limb on the movable plate 60A and applying the ultrasound probe 42 to the measurement target portion from the lower side of the movable plate 60A through the sensor facing region 61A in a state where the movable plate 60A is pulled out.

In FIG. 18, a circle represents that the ultrasound image has a good image quality (visibility), and a triangle represents that the ultrasound image has a slightly good image quality. A cross represents that the ultrasound image has a poor image quality, and the “-” represents not evaluated.

By the evaluation indicated in FIG. 18, it was confirmed that the materials by which good visibility was achieved for the median nerve and sural nerve in the ultrasound images, were Acrylonitrile Butadiene Styrene (ABS) resin, polystyrene, and polycarbonate, which are 1 mm thick. In the case of ABS resin, even when the material was 2 mm thick, it was confirmed that the visibility of the median nerve and sural nerve in the ultrasound image was good.

Note that the movable plate 60A is not limited to the structure described above, as long as an ultrasound image of the measurement target portion of the subject P mounted on the movable plate 60A can be acquired. For example, a slit or hole may be provided in the sensor facing region 61A of the movable plate 60A at predetermined intervals. Furthermore, when the area of the measurement target portion is small, an opening may be provided in the sensor facing region 61A in accordance with the measurement target portion.

Further, when the area of the measurement target portion is small and the strength of the sensor facing region 61A is not required, the sensor facing region 61A may be formed of a material having an acoustic impedance close to the acoustic impedance of the human body (e.g., a gel material), rather than a solid material such as a resin.

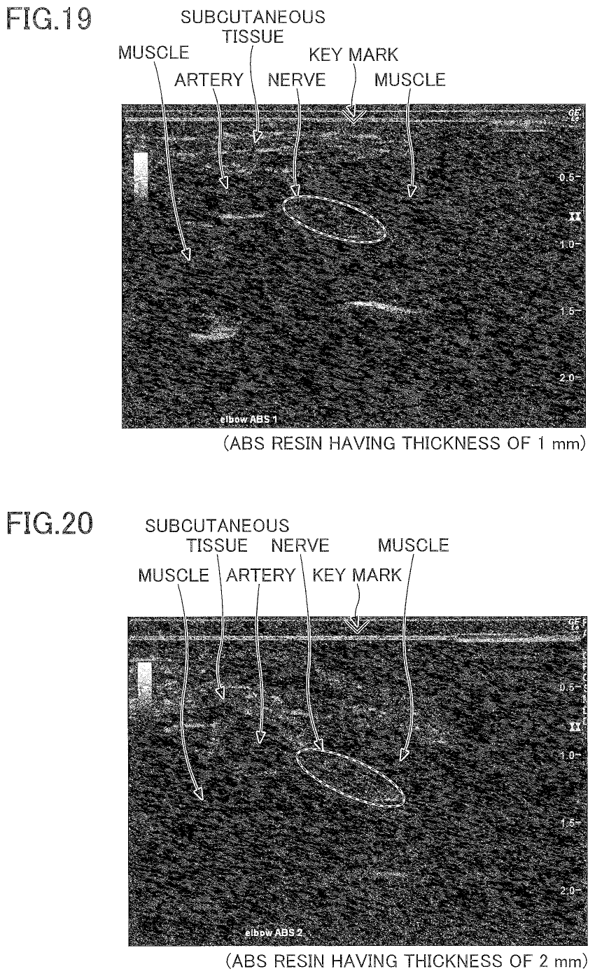

FIGS. 19 to 24 are diagrams illustrating examples of ultrasound images of a measurement target portion acquired through the sensor facing region 61A formed of the materials indicated in FIG. 18. The top center of each ultrasound image displays a key mark indicating the center position of the ultrasound probe 42.

As described in FIG. 12, an ultrasound image of a measurement target portion of a subject P is acquired by the ultrasound probe 42 which is placed in a space formed under the sensor facing region 61A of the movable plate 60A in a state where the movable plate 60A has been moved to the outside of the protruding part 21. In each ultrasound image, a nerve is present at the position indicated by the white dashed line circle.

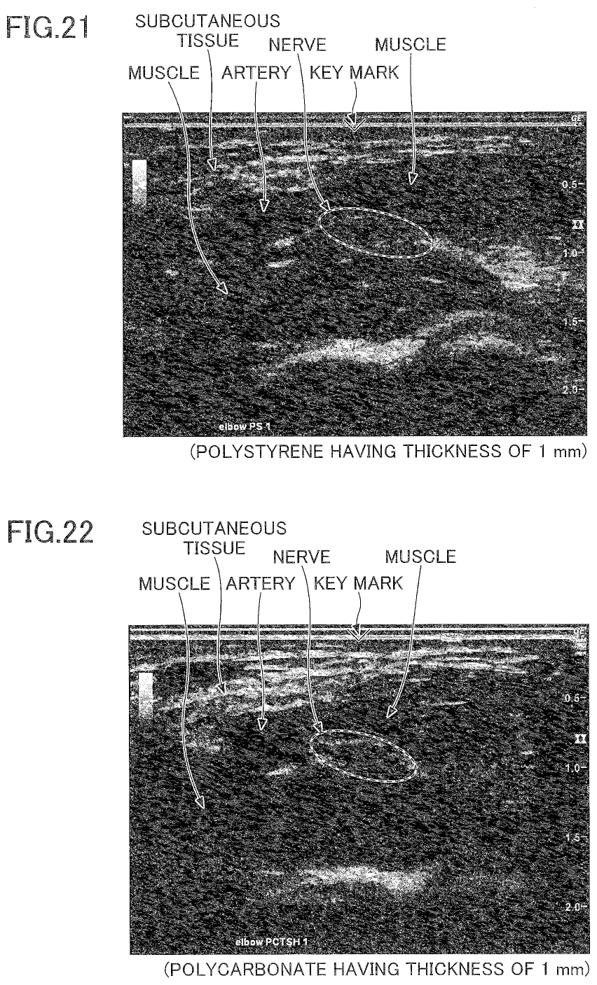

FIG. 19 illustrates an ultrasound image acquired through the sensor facing region 61A formed of ABS resin that is 1 mm thick. FIG. 20 illustrates an ultrasound image acquired through the sensor facing region 61A formed of ABS resin that is 2 mm thick. FIG. 21 illustrates an ultrasound image acquired through the sensor facing region 61A formed of polystyrene that is 1 mm thick.

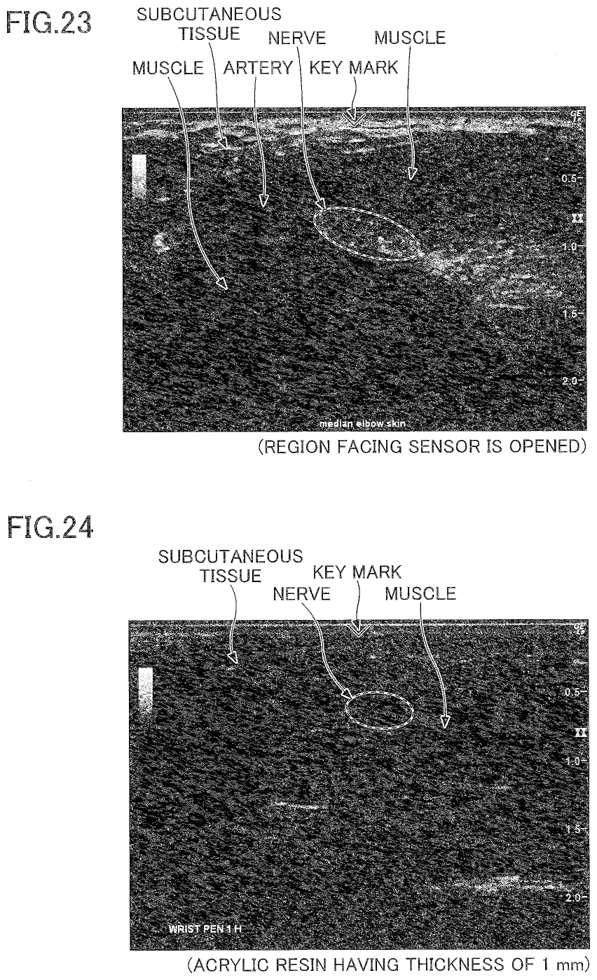

FIG. 22 illustrates an ultrasound image acquired through the sensor facing region 61A formed of polycarbonate that is 1 mm thick. FIG. 23 illustrates an ultrasound image acquired by opening the portion corresponding to the measurement target portion in the sensor facing region 61A. FIG. 24 illustrates an ultrasound image acquired through the sensor facing region 61A formed of acrylic resin that is 1 mm thick.

FIGS. 19 to 22 are ultrasound images with good evaluation results (circles) as indicated in FIG. 18, and the nerves indicated by the dashed line white circles are clearly visible. With the ultrasound image acquired by forming an opening in the sensor facing region 61A of FIG. 23, it is possible to acquire a clear image of the nerve because the ultrasound probe 42 is directly applied to the measurement target portion. On the other hand, for example, in the ultrasound image acquired through the acrylic resin that is 1 mm thick illustrated in FIG. 24, the nerve indicated by the dashed line white circle cannot be appreciably identified.

As described above, in the present embodiment, the same effect as in the above-described embodiment can be obtained. In the present embodiment, an ultrasound image of a measurement target portion having good image quality (visibility) can be acquired while maintaining the strength (stiffness) of the movable plate 60A. Accordingly, based on the ultrasound image and the image acquired by the camera 300 illustrated in FIG. 8 or the like, the positional relationship between the position of the nerve and the position of each magnetic sensor can be reliably detected, and the current distribution of the measurement target portion can be estimated from the biomagnetic field data based on the positional relationship. That is, by using an ultrasound image including a nerve image, it is possible to estimate the electrical current distribution in a living body from biomagnetic field data of peripheral nerves.

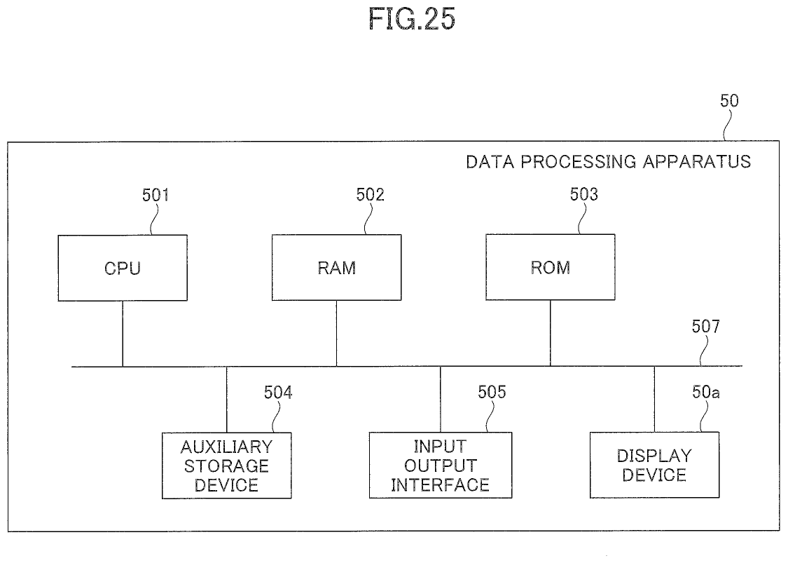

FIG. 25 is a block diagram illustrating an example of a hardware configuration of the data processing apparatus 50 of FIGS. 8, 9, and 16. For example, the data processing apparatus 50 includes a CPU 501, a RAM 502, a ROM 503, an auxiliary storage device 504, an input output interface 505, and the display device 50a interconnected by a bus 507.

The CPU 501 controls the overall operation of the data processing apparatus 50. The CPU 501 implements the functions of the position information acquiring unit 52, the positional relationship acquiring unit 54, and the current estimating unit 56 by executing a bioelectric current estimation program stored in the ROM 503 or the auxiliary storage device 504. The CPU 501 may control the operation of the biomagnetic measurement system 2100, such as the magnetic measurement apparatus 210 and the ultrasonic measurement apparatus 40.

The RAM 502 is used as a work area of the CPU 501 and stores the bioelectric current estimation program and various parameters such as the Z position and the XY position. The ROM 503 stores a bioelectric current estimation program.

The auxiliary storage device 504 is a storage device such as a Solid State Drive (SSD) or a Hard Disk Drive (HDD). For example, the auxiliary storage device 504 stores a control program such as an operating system (OS) for controlling the operation of the data processing apparatus 50, an ultrasound image, morphological image data, various parameters, and the like.

The input output interface 505 is connected to a mouse, keyboard, or the like. The input output interface 505 may include a communication interface for communicating with other devices. The display device 50a displays windows and operation windows for displaying the current waveform illustrated in FIG. 5. The display device 50a may display an ultrasound image illustrated FIG. 14A or a diagram illustrating the positional relationship between the nerve and the sensor array 211 illustrated in FIG. 15.

According to one embodiment of the present invention, the positional relationship between a sensor for measuring the biomagnetic field and a nerve can be acquired by using a nerve image included in an image of a measurement target region to estimate the neural activity current generated in association with the neural activity of a subject.

According to another embodiment of the present invention, the positional relationship between the position of a nerve and the position of a magnetic sensor at the time of measuring a magnetic field can be identified by using an ultrasound image, so that the current distribution can be estimated from the biomagnetic field data.

The bioelectric current estimation method, the bioelectric current estimation apparatus, the biomagnetic measurement apparatus, and the biomagnetic measurement system are not limited to the specific embodiments described in the detailed description, and variations and modifications may be made without departing from the spirit and scope of the present invention.

What is claimed is:

1. A bioelectric current estimation method comprising:
acquiring position information of a nerve in a measurement target region of a subject for which magnetic data is measured with a magnetic sensor, the position information of the nerve being acquired based on a nerve image included in a morphological image of the measurement target region;
acquiring a positional relationship between a position of the nerve and a position of the magnetic sensor, based on the acquired position information of the nerve and position information of the magnetic sensor when the magnetic sensor is positioned to face the measurement target region; and
estimating a neural activity current, which is generated in association with neural activity of the subject, based on the acquired positional relationship and the magnetic data of the measurement target region measured by the magnetic sensor.

2. The bioelectric current estimation method according to claim 1, wherein the position information of the nerve includes a distance in a depth direction from a surface of the measurement target region to the nerve when the magnetic sensor is positioned to face the measurement target region.

3. The bioelectric current estimation method according to claim 2, wherein the position information of the nerve includes a two-dimensional position of the nerve on the surface of the measurement target region.

4. The bioelectric current estimation method according to claim 1, wherein the position information of the magnetic sensor includes positions of a plurality of sensors arranged in an array included in the magnetic sensor.

5. The bioelectric current estimation method according to claim 4, wherein a distance in a depth direction from a surface of the measurement target region to the nerve when the magnetic sensor is positioned to face the measurement target region, is corrected in accordance with the measurement target region having a shape corresponding to a cover member having a curved shape covering leading ends of the plurality of sensors.

6. The bioelectric current estimation method according to claim 1, wherein the morphological image including the nerve image is an ultrasound image.

7. The bioelectric current estimation method according to claim 1, wherein the morphological image including the nerve image is a magnetic resonance (MR) image.

8. A bioelectric current estimation apparatus comprising:
a position information acquirer configured to acquire position information of a nerve in a measurement target region of a subject for which magnetic data is measured with a magnetic sensor, the position information of the nerve being acquired based on a nerve image included in a morphological image of the measurement target region;
a positional relationship acquirer configured to acquire a positional relationship between a position of the nerve and a position of the magnetic sensor, based on the acquired position information of the nerve and position information of the magnetic sensor when the magnetic sensor is positioned to face the measurement target region; and
an estimator configured to estimate a neural activity current, which is generated in association with neural activity of the subject, based on the acquired positional relationship and the magnetic data of the measurement target region measured by the magnetic sensor.

9. A biomagnetic measurement system comprising:
a biomagnetic measurement apparatus including a magnetic sensor configured to measure magnetic data of a subject; and
a bioelectric current estimation apparatus configured to estimate a neural activity current, which is generated in association with neural activity of the subject, based on the magnetic data of the subject measured by the magnetic sensor and a nerve image of the subject, wherein the bioelectric current estimation apparatus includes:
a position information acquirer configured to acquire position information of a nerve in a measurement target region of the subject for which the magnetic data is measured, the position information of the nerve being acquired based on the nerve image included in a morphological image of the measurement target region of the subject;
a positional relationship acquirer configured to acquire a positional relationship between a position of the nerve and a position of the magnetic sensor, based on the acquired position information of the nerve and position information of the magnetic sensor when the magnetic sensor is positioned to face the measurement target region; and
an estimator configured to estimate the neural activity current based on the acquired positional relationship and the magnetic data of the measurement target region measured by the magnetic sensor.

10. A biomagnetic measurement apparatus comprising:
a sensor housing configured to house a magnetic sensor;
a plate member, which is transparent, separably disposed on an opposing surface included in the sensor housing, the opposing surface facing a detector included in the magnetic sensor; and
a current estimator configured to estimate a current distribution of a measurement target portion from biomagnetic data of the measurement target portion measured by the magnetic sensor, based on a positional relationship between a nerve in the measurement target portion and the magnetic sensor in a state where the plate member is separated from the opposing surface, the positional relationship being obtained from position information of the nerve in the measurement target portion acquired by an ultrasound probe applied to the plate member that is in contact with the measurement target portion and an image including the ultrasound probe and the plate member when the position information of the nerve is acquired.

11. The biomagnetic measurement apparatus according to claim 10, further comprising:
a guide member that is mounted to the sensor housing and configured to guide the plate member that moves along the opposing surface.

12. The biomagnetic measurement apparatus according to claim 10, further comprising:
a position information acquirer configured to acquire the position information representing a position of the nerve on the opposing surface and a depth of the nerve from the opposing surface when the plate member in contact with the measurement target portion is facing the opposing surface, the position information being acquired based on a nerve image included in a morphological image of the measurement target portion acquired by the ultrasound probe and the image including the ultrasound probe and the plate member captured from above or below the opposing surface when the morphological image is acquired; and
a positional relationship acquirer configured to acquire the positional relationship between the nerve and the magnetic sensor based on the position information of the nerve acquired by the position information acquirer and position information of the magnetic sensor with respect to the plate member facing the opposing surface, wherein
the current estimator estimates a neural activity current based on the positional relationship acquired by the positional relationship acquirer and the biomagnetic data.

13. A biomagnetic measurement apparatus comprising:
a sensor housing configured to house a magnetic sensor;
a plate member, which is transparent, separably disposed on an opposing surface included in the sensor housing, the opposing surface facing a detector included in the magnetic sensor, the plate member being provided with a plurality of marker coils; and
a current estimator configured to estimate a current distribution of a measurement target portion from biomagnetic data of the measurement target portion measured by the magnetic sensor, based on a positional relationship between a nerve in the measurement target portion and the magnetic sensor in a state where the plate member is separated from the opposing surface, the positional relationship being obtained from position information of the nerve in the measurement target portion acquired by an ultrasound probe applied to the plate member that is in contact with the measurement target portion and a positional relationship between the ultrasound probe and the plate member acquired based on a magnetic field generated from the plurality of marker coils measured by a magnetic sensor provided in the ultrasound probe.

14. The biomagnetic measurement apparatus according to claim 10, wherein at least a part of the plate member is formed of a material by which the position information of the nerve in a subject can be acquired by the ultrasound probe.

15. The biomagnetic measurement apparatus according to claim 10, wherein at least a part of the plate member is provided with an opening in a region from which the position information of the nerve in a subject is acquired by the ultrasound probe.

16. A biomagnetic measurement system comprising:
a biomagnetic measurement apparatus including a dewar including a sensor housing configured to house a magnetic sensor;
an ultrasonic measurement apparatus including an ultrasound probe; and
a camera installed above or below the sensor housing, wherein the biomagnetic measurement apparatus includes:
a plate member, which is transparent, separably disposed on an opposing surface included in the sensor housing, the opposing surface facing a detector included in the magnetic sensor; and
a current estimator configured to estimate a current distribution of a measurement target portion from biomagnetic data of the measurement target portion measured by the magnetic sensor, based on a positional relationship between a nerve in the measurement target portion and the magnetic sensor in a state where the plate member is separated from the opposing surface, the positional relationship being obtained from position information of the nerve in the measurement target portion acquired by the ultrasound probe applied to the plate member that is in contact with the measurement target portion and an image, which is captured by the camera, including the ultrasound probe and the plate member when the position information of the nerve is acquired.
